(19) United States
(12) Patent Application Publication
(10) Pub. No.: US 2021/0054068 A1
(43) Pub. Date: Feb. 25, 2021

(54) DETECTION OF PANCREATIC CANCER WITH AN ENGINEERED ANTI-THY1 SINGLE-CHAIN ANTIBODY

(71) Applicant: The Board Of Trustees Of The Leland Stanford Junior University, Stanford, CA (US)

(72) Inventors: Lotfi Abou-Elkacem, Houston, TX (US); Juergen Karl Willmann, Stanford, CA (US)

(21) Appl. No.: 16/304,771

(22) PCT Filed: Jun. 5, 2017
(86) PCT No.: PCT/US17/35989

§ 371 Date: Nov. 27, 2018

Related U.S. Application Data
(60) Provisional application No. 62/347,345, filed on Jun. 8, 2016.

(51) Int. Cl. C07K 16/28; A61K 49/22; A61K 51/10; A61K 47/68; A61K 45/06; A61K 39/395; G01N 33/574

(52) U.S. Cl. CPC C07K 16/2803; A61K 49/221; A61K 49/223; A61K 51/1057; A61K 47/6859; C07K 2317/92; A61K 39/39558; G01N 33/57438; C07K 2317/33; C07K 2317/622; A61K 45/06

(57) ABSTRACT

Compositions and methods for detection and diagnosis of pancreatic cancer are disclosed. An engineered anti-thymocyte differentiation antigen 1 (Thy1) single-chain antibody and bioconjugates thereof can be used for detecting and diagnosing pancreatic adenocarcinoma. The anti-Thy1 single-chain antibody selectively binds to the Thy1 antigen, which is overexpressed on pancreatic tumor neovasculature and precancerous lesions, and is capable of detecting pancreatic cancer even at the earliest stages of the disease. Thy1-targeted diagnostic agents can be produced by conjugation of the anti-Thy1 single-chain antibody to various diagnostic agents, such as contrast agents, photoactive agents, or detectable labels that are useful for detection and medical imaging of pancreatic tumors.

### CROSS REFERENCE

This application claims benefit of U.S. Provisional Patent Application No. 62/347,345, filed Jun. 8, 2016, which application is incorporated herein by reference in its entirety.

### STATEMENT REGARDING FEDERALLY SPONSORED RESEARCH OR DEVELOPMENT

This invention was made with Government support under contract CA151459 awarded by the National Institutes of Health. The Government has certain rights in the invention.

### TECHNICAL FIELD

Methods of diagnosing and treating pancreatic cancer and medical imaging of pancreatic tumors are provided. In particular, an engineered anti-thymocyte differentiation antigen 1 (Thy1, CD90) single-chain antibody and bioconjugates thereof are provided that can be used for detection of pancreatic adenocarcinoma even at the earliest stages of the disease.

### BACKGROUND

Pancreatic ductal adenocarcinoma (PDAC) is a highly lethal form of cancer. While patient survival is highly dependent upon tumor stage, most patients already have advanced disease at the time of diagnosis. The incidence of PDAC is on the rise and PDAC is projected to become the second most common cause of cancer death by 2020. Because of the advanced state of the disease at diagnosis, only 15-20% of patients with PDAC are candidates for potentially curative surgical resection. While unpredictable and vague clinical symptoms related to the disease are factors in the delay in diagnosis, there is a lack of specific and sensitive blood biomarker and imaging tests to detect the disease early. Unfortunately, current chemotherapy and radiotherapy approaches offer only moderate survival benefits.

Currently used imaging techniques, including abdominal computed tomography, magnetic resonance imaging, and cholangiopancreatography, as well as transabdominal and endoscopic ultrasound, can be non-specific in detecting early-stage PDAC. At this time, the best available and inexpensive diagnostic test, which is routinely performed in patients at high risk for PDAC, is an endoscopic ultrasound (EUS). However, EUS has low specificity, poor inter-observer reliability, and difficulty in differentiating chronic pancreatitis from PDAC. Patients diagnosed at stage I can survive 5 years in up to 30-40% of cases, yet the overall median survival of patients after diagnosis is only 4 to 6 months, reflecting the advanced stage of the disease at diagnosis of most patients. Therefore, detection of PDAC at early stages would significantly improve survival of patients with PDAC.

### SUMMARY

An engineered anti-thymocyte differentiation antigen 1 (Thy1) single-chain antibody and bioconjugates thereof are provided that can be used for detection, diagnosis, and medical imaging of pancreatic adenocarcinoma even at the earliest stages of the disease.

In some embodiments, an anti-Thy1 single-chain antibody is provided comprising an amino acid sequence of SEQ ID NO:1 and variants thereof comprising an amino acid sequence displaying at least about 80-100% sequence identity to the amino acid sequence of SEQ ID NO:1, including any percent identity within this range, such as 81, 82, 83, 84, 85, 86, 87, 88, 89, 90, 91, 92, 93, 94, 95, 96, 97, 98, 99% sequence identity thereto, for example comprising CDR sequences of SEQ ID NO:1, and Thy1-binding fragments thereof that bind to the same epitope as or compete for binding to Thy1 with a full-length anti-Thy1 single-chain antibody described herein. In one embodiment, the single chain antibody binds to human Thy1. In another embodiment, the single chain antibody binds to human Thy1 with a dissociation constant (Kd) of less than or equal to 3 nM.

In certain embodiments, a Thy1-targeted imaging agent comprising an anti-Thy1 single chain antibody described herein is conjugated to a diagnostic agent. The diagnostic agent can be, for example, an isotopic label, a fluorescent label, a chemiluminescent label, a bioluminescent label, a paramagnetic ion, an enzyme, a contrast agent (e.g., ultrasound contrast agent, a magnetic resonance imaging (MRI) contrast agent, or a radiocontrast agent), or a photoactive agent.

Exemplary fluorescent labels include fluorescein derivatives, rhodamine derivatives, coumarin derivatives, cyanine derivatives, acridine derivatives, squaraine derivatives, naphthalene derivatives, oxadiazol derivatives, anthracene derivatives, pyrene derivatives, oxazine derivatives, arylmethine derivatives, and tetrapyrrole derivatives. In addition, the fluorescent label may comprise a fluorescent protein, such as, but not limited to, green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), TagRFP, Dronpa, Padron, mApple, mCherry, rsCherry, and rsCherryRev.

Isotopic labels may comprise radioactive isotopes (e.g., gamma-emitters, beta-emitters, and positron-emitters) or non-radioactive isotopes (e.g., stable trace isotopes), such as, but not limited to, ³H, ²H, ¹²⁰I, ¹²³I, ¹²⁴I, ¹²⁵I, ¹³¹I, ³⁵S, ¹¹C, ¹³C, ¹⁴C, ³²P, ¹⁵N, ¹³N, ¹¹⁰In, ¹¹¹In, ¹⁷⁷Lu, ¹⁸F, ⁵²Fe, ⁶²Cu, ⁶⁴Cu, ⁶⁷Cu, ⁶⁷Ga, ⁶⁸Ga, ⁸⁶Y, ⁹⁰Y, ⁸⁹Zr, ^{94m}Tc, ⁹⁴Tc, ^{99m}Tc, ¹⁵⁴Gd, ¹⁵⁵Gd, ¹⁵⁶Gd, ¹⁵⁷Gd, ¹⁵⁸Gd, ¹⁵O, ¹⁸⁶Re, ¹⁸⁸Re, ⁵¹M, ^{52m}Mn, ⁵⁵Co, ⁷²As, ⁷⁵Br, ⁷⁶Br, ^{82m}Rb, and ⁸³Sr.

Exemplary paramagnetic ions include chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and erbium (III).

In another embodiment, the invention includes a Thy1-targeted imaging agent comprising an anti-Thy1 single chain antibody, described herein, conjugated to a contrast agent. For example, the contrast agent may be an ultrasound contrast agent (e.g., a microbubble), a magnetic resonance imaging (MRI) contrast agent, or a radiocontrast agent.

In another embodiment, the anti-Thy1 single chain antibody is conjugated to an anti-cancer therapeutic agent. Exemplary therapeutic agents include a cytotoxic agent, a drug, a toxin, a nuclease, a hormone, a therapeutic enzyme, an immunomodulator, a pro-apoptotic agent, an angiogenesis inhibitor, a boron compound, a photoactive agent, and a radioisotope. In certain embodiments, the anti-Thy1 single chain antibody is conjugated to both a diagnostic agent and a therapeutic agent (i.e., Thy1-targeted theranostic agent).

In another embodiment, the invention includes a composition comprising an anti-Thy1 single-chain antibody bioconjugate described herein (e.g., a Thy1-targeted imaging agent, therapeutic agent, or theranostic agent). The composition may further comprise a pharmaceutically acceptable excipient. In certain embodiments, the composition further comprises one or more anti-cancer therapeutic agents.

In another aspect, the invention includes a method of detecting pancreatic cancer or precancerous lesions, the method comprising: a) administering a detectably effective amount of a Thy1-targeted imaging agent, described herein, to a patient suspected of having pancreatic cancer, under conditions wherein the Thy1-targeted imaging agent binds to Thy1 present on pancreatic tumor neovasculature, cancerous cells, or precancerous lesions, if present, in the patient; and b) detecting the Thy1-targeted imaging agent bound to the pancreatic tumor neovasculature, cancerous cells, or precancerous lesions, if present, by imaging pancreatic tissue of the patient. In one embodiment, the pancreatic cancer is pancreatic ductal adenocarcinoma. In another embodiment, the patient is human.

In certain embodiments, imaging of pancreatic tissue is performed using a method selected from the group consisting of ultrasound imaging (UI), positron emission tomography (PET), single photon emission computed tomography (SPECT), magnetic resonance imaging (MRI), computed tomography (CT), optical imaging (OI), photoacoustic imaging (PI), and fluorescence imaging.

The methods can be used for determining the prognosis of the patient. Detection of a precancerous lesion indicates the patient is at risk of developing pancreatic cancer. Detection of increased levels of Thy1 antigen on the surface of pancreatic tumor neovasculature and cancerous cells is associated with tumor growth and cancer progression. Following such detection a patient may be treated for the cancer if determined to be present.

In another aspect, the invention includes a method of imaging pancreatic tissue of a patient suspected of having pancreatic cancer, the method comprising: a) contacting pancreatic tissue of the patient with a detectably effective amount of a Thy1-targeted imaging agent described herein under conditions wherein the Thy1-targeted imaging agent binds to Thy1 present on any pancreatic tumor neovasculature, cancerous cells, or precancerous lesions, if present in the pancreatic tissue; and b) imaging pancreatic tissue of the patient, wherein detection of increased binding of the Thy1-targeted imaging agent to the pancreatic tissue of the patient compared to a control indicates that the patient has pancreatic cancer or precancerous lesions. The pancreatic tissue may be contacted with the Thy1-targeted imaging agent either in vivo or in vitro.

In certain embodiments, imaging of pancreatic tissue is performed using a method selected from the group consisting of ultrasound imaging (UI), positron emission tomography (PET), single photon emission computed tomography (SPECT), magnetic resonance imaging (MRI), computed tomography (CT), optical imaging (OI), photoacoustic imaging (PI), and fluorescence imaging.

Included is a method of monitoring progression of pancreatic cancer in a patient, the method comprising: imaging pancreatic tissue of the patient according to a method described herein, wherein a first image is obtained at a first time point and a second image is obtained later at a second time point, wherein detection of increased binding of the Thy1-targeted imaging agent to the pancreatic tissue of the patient at the second time point compared to the first time point indicates that the patient is worsening, and detection of decreased binding of the Thy1-targeted imaging agent to the pancreatic tissue of the patient at the second time point compared to the first time point indicates that the patient is improving. Increased binding of the Thy1-targeted imaging agent to the pancreatic tissue of the patient may be caused, for example, by growth of a pancreatic tumor or the presence of more pancreatic tumors or lesions at the second time point, which can be determined by inspection of the images. Alternatively, decreased binding of the Thy1-targeted imaging agent to the pancreatic tissue of the patient may be caused, for example, by tumor shrinkage or the presence of fewer pancreatic tumors or lesions.

In some embodiments, a method for evaluating the effect of an agent for treating pancreatic cancer in a patient is provided, the method comprising: imaging pancreatic tissue of the patient according to a method described herein before and after the patient is treated with said agent, wherein detection of increased binding of the Thy1-targeted imaging agent to the pancreatic tissue of the patient (e.g., from tumor growth or increase in number of tumors or cancer cells) after the patient is treated with said agent compared to before the patient is treated with said agent indicates that the patient is worsening, and decreased binding of the Thy1-targeted imaging agent to the pancreatic tissue of the patient (e.g., from reduction in tumor size or reduction in the number of cancer cells) after the subject is treated with said agent compared to before the patient is treated with said agent indicates that the patient is improving.

A method for monitoring the efficacy of a therapy for treating pancreatic cancer in a patient is provided, the method comprising: imaging pancreatic tissue of the patient according to a method described herein before and after the subject undergoes said therapy, wherein detection of increased binding of the Thy1-targeted imaging agent to the pancreatic tissue of the patient (e.g., from tumor growth or increase in number of tumors or cancer cells) after the patient undergoes said therapy compared to before the patient undergoes said therapy indicates that the patient is worsening, and decreased binding of the Thy1-targeted imaging agent to the pancreatic tissue of the patient (e.g., from reduction in tumor size or reduction in the number of cancer cells) after the patient undergoes said therapy compared to before the patient undergoes said therapy indicates that the patient is improving. The patient may be treated in accordance with the findings, where, for example a patient that is not improving may be switched to alternative treatment; or a patient that is improving may be maintained on current therapy.

In another aspect, the invention includes a kit comprising an anti-Thy1 single chain antibody, described herein, or a bioconjugate thereof (e.g., Thy1-targeted imaging agent, therapeutic agent, or theranostic agent) and instructions for using the kit to diagnose and/or treat pancreatic cancer.

A method of treating a patient suspected of having pancreatic cancer is provided, the method comprising: a) receiving information regarding whether or not pancreatic cancer was detected in the patient using a Thy1-targeted imaging agent according to a method described herein; and b) administering anti-cancer therapy to the subject if pancreatic cancer was detected in the patient. In certain embodiments, the anti-cancer therapy comprises surgery, radiation therapy, chemotherapy, hormonal therapy, immunotherapy, or biologic therapy, or any combination thereof.

Isolated polynucleotide(s) encoding an anti-Thy1 single chain antibody described herein are provided. A recombinant polynucleotide may comprise the polynucleotide encoding the anti-Thy1 single chain antibody operably linked to a promoter. In certain embodiments, the recombinant polynucleotide comprises a polynucleotide encoding an anti-Thy1 single-chain antibody comprising an amino acid sequence of SEQ ID NO:1 or a variant thereof comprising an amino acid sequence displaying at least about 80-100% sequence identity to the amino acid sequence of SEQ ID NO:1, including any percent identity within this range, such as 81, 82, 83, 84, 85, 86, 87, 88, 89, 90, 91, 92, 93, 94, 95, 96, 97, 98, 99% sequence identity thereto, or a Thy1-binding fragment thereof that binds to the same epitope as or compete for binding to Thy1 with a full-length anti-Thy1 single-chain antibody described herein.

An isolated host cell comprising a recombinant polynucleotide encoding an anti-Thy1 single chain antibody operably linked to a promoter is also provided.

A method for producing an anti-Thy1 single-chain antibody described herein is provided, the method comprising: a) transforming a host cell with a recombinant polynucleotide comprising a polynucleotide encoding the anti-Thy1 single-chain antibody operably linked to a promoter; b) culturing the transformed host cell under conditions whereby the anti-Thy1single-chain antibody is expressed; and c) isolating the anti-Thy1single-chain antibody from the host cell.

These and other embodiments will readily occur to those of skill in the art in view of the disclosure herein.

### DETAILED DESCRIPTION

Conventional methods of medicine, pharmacology, chemistry, biochemistry, recombinant DNA techniques and immunology may be used, as known to one of skill in the art. Such techniques are explained fully in the literature. See, e.g., Pancreatic Cancer (Recent Results in Cancer Research, H. Riess, A. Goerke, and H. Oettle eds., Springer, 2008 edition); Pancreatic Cancer (Contemporary Issues in Cancer Imaging, J. Heiken ed., Cambridge University Press, 2009); A. B. Wolbarst et al. Medical Imaging: Essentials for Physicians (Wiley-Blackwell, 2013); Handbook of Experimental Immunology, Vols. I-IV (D. M. Weir and C. C. Blackwell eds., Blackwell Scientific Publications); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.).

All publications, patents and patent applications cited herein, whether supra or infra, are hereby incorporated by reference in their entireties.

### I. Definitions

The following terms are employed, and are intended to be defined as indicated below.

It must be noted that, as used in this specification and the appended claims, the singular forms “a”, “an” and “the” include plural referents unless the content clearly dictates otherwise. Thus, for example, reference to “an antibody” includes a mixture of two or more antibodies, and the like.

The term “about”, particularly in reference to a given quantity, is meant to encompass deviations of plus or minus five percent.

The terms “tumor,” “cancer” and “neoplasia” are used interchangeably and refer to a cell or population of cells whose growth, proliferation or survival is greater than growth, proliferation or survival of a normal counterpart cell, e.g. a cell proliferative, hyperproliferative or differentiative disorder. Typically, the growth is uncontrolled. The term “malignancy” refers to invasion of nearby tissue. The term “metastasis” or a secondary, recurring or recurrent tumor, cancer or neoplasia refers to spread or dissemination of a tumor, cancer or neoplasia to other sites, locations or regions within the subject, in which the sites, locations or regions are distinct from the primary tumor or cancer. Neoplasia, tumors and cancers include benign, malignant, metastatic and non-metastatic types, and include any stage (I, II, III, IV or V) or grade (G1, G2, G3, etc.) of neoplasia, tumor, or cancer, or a neoplasia, tumor, cancer or metastasis that is progressing, worsening, stabilized or in remission

The terms “subject,” “individual,” and “patient,” are used interchangeably herein and refer to any mammalian subject for whom diagnosis, prognosis, treatment, or therapy is desired, particularly humans. Other subjects may include cattle, dogs, cats, guinea pigs, rabbits, rats, mice, horses, and so on. In some cases, the methods of the invention find use in experimental animals, in veterinary application, and in the development of animal models for disease, including, but not limited to, rodents including mice, rats, and hamsters; and primates.

The terms “quantity,” “amount,” and “level” are used interchangeably herein and may refer to an absolute quantification of a molecule or an analyte (e.g., Thy1), or to a relative quantification of a molecule or analyte, i.e., relative to another value such as relative to a reference value as taught herein, or to a range of values for the molecule or analyte. These values or ranges can be obtained from a single patient or from a group of patients.

The term “antibody” encompasses polyclonal and monoclonal antibody preparations, as well as preparations including hybrid antibodies, altered antibodies, chimeric antibodies and, humanized antibodies, as well as: hybrid (chimeric) antibody molecules (see, for example, Winter et al. (1991) Nature 349:293-299; and U.S. Pat. No. 4,816,567); F(ab′)₂ and F(ab) fragments; Fᵥ molecules (noncovalent heterodimers, see, for example, Inbar et al. (1972) Proc Natl Acad Sci USA 69:2659-2662; and Ehrlich et al. (1980) Biochem 19:4091-4096); single-chain Fv molecules (sFv) (see, e.g., Huston et al. (1988) Proc Natl Acad Sci USA 85:5879-5883); dimeric and trimeric antibody fragment constructs; minibodies (see, e.g., Pack et al. (1992) Biochem 31:1579-1584; Cumber et al. (1992) J Immunology 149B:120-126); humanized antibody molecules (see, e.g., Riechmann et al. (1988) Nature 332:323-327; Verhoeyan et al. (1988) Science 239:1534-1536; and U.K. Patent Publication No. GB 2,276,169, published 21 Sep. 1994); and, any functional fragments obtained from such molecules, wherein such fragments retain specific-binding properties of the parent antibody molecule.

A “single-chain antibody,” “single chain variable fragment,” or “scFv” comprises an antibody heavy chain variable domain (VH) and a light-chain variable domain (VL) joined together by a flexible peptide linker. The peptide linker is typically 10-25 amino acids in length. Single-chain antibodies retain the antigen-binding properties of natural full-length antibodies, but are smaller that natural intact antibodies or Fab fragments because of the lack of an Fc domain.

“Immunoassay” is an assay that uses an antibody (e.g., a single-chain antibody) to specifically bind an antigen (e.g., Thy1). The immunoassay is characterized by the use of specific binding properties of a particular antibody to isolate, target, and/or quantify the antigen. An immunoassay for detection of an antigen may utilize one antibody or several antibodies. Immunoassay protocols may be based, for example, upon competition, direct reaction, or sandwich type assays using, for example, a labeled antibody. The labels may be, for example, fluorescent, chemiluminescent, or radioactive. Alternatively, the antibody may be conjugated to a diagnostic agent, such as a contrast agent or photoactive agent that is useful for biomedical imaging (e.g., ultrasound, MRI, or CT)

The phrase “specifically (or selectively) binds” or “specifically (or selectively) immunoreactive with,” when referring to binding of an antibody to a protein (or peptide), refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present. Specific binding of an antibody under such conditions may require an antibody that is selected for its specificity for a particular protein. Typically specific or selective binding will be at least twice the background signal or noise and more typically more than 10 to 100 times background.

An antibody binds “essentially the same epitope” as a reference antibody, when the two antibodies recognize identical or sterically overlapping epitopes. The most widely used and rapid methods for determining whether two antibodies bind to identical or sterically overlapping epitopes are competition assays, which can be configured in all number of different formats, using either labeled antigen or labeled antibody. Usually, the antigen is immobilized on a substrate, and the ability of unlabeled antibodies to block the binding of labeled antibodies is measured using radioactive isotopes or enzyme labels.

A “Thy1-targeted diagnostic agent” or “Thy1-targeted imaging agent” refers to an anti-Thy1 single-chain antibody that is detectably labeled or conjugated to a diagnostic or detection agent. For example, an anti-Thy1 single-chain antibody can be directly conjugated to a detectable diagnostic agent and used in detection, diagnosis, or medical imaging of pancreatic cancer.

As used herein, the terms “detection agent”, “diagnostic agent”, and “detectable label” refer to a molecule or substance capable of detection, including, but not limited to, fluorescers, chemiluminescers, chromophores, bioluminescent proteins, enzymes, enzyme substrates, enzyme cofactors, enzyme inhibitors, isotopic labels, semiconductor nanoparticles, dyes, metal ions, metal sols, ligands (e.g., biotin, streptavidin or haptens) and the like. The term “fluorescer” refers to a substance or a portion thereof which is capable of exhibiting fluorescence in the detectable range. Particular examples of labels which may be used in the practice of the invention include, but are not limited to, SYBR green, SYBR gold, a CAL Fluor dye such as CAL Fluor Gold 540, CAL Fluor Orange 560, CAL Fluor Red 590, CAL Fluor Red 610, and CAL Fluor Red 635, a Quasar dye such as Quasar 570, Quasar 670, and Quasar 705, an Alexa Fluor such as Alexa Fluor 350, Alexa Fluor 488, Alexa Fluor 546, Alexa Fluor 555, Alexa Fluor 594, Alexa Fluor 647, and Alexa Fluor 784, a cyanine dye such as Cy 3, Cy3.5, Cy5, Cy5.5, and Cy7, fluorescein, 2′, 4′, 5′, 7′-tetrachloro-4-7-dichlorofluorescein (TET), carboxyfluorescein (FAM), 6-carboxy-4′,5′-dichloro-2′,7′-dimethoxyfluorescein (JOE), hexachlorofluorescein (HEX), rhodamine, carboxy-X-rhodamine (ROX), tetramethyl rhodamine (TAMRA), FITC, dansyl, umbelliferone, dimethyl acridinium ester (DMAE), Texas red, luminol, and quantum dots, enzymes such as alkaline phosphatase (AP), beta-lactamase, chloramphenicol acetyltransferase (CAT), adenosine deaminase (ADA), aminoglycoside phosphotransferase (neo^{r}, G418^{r}) dihydrofolate reductase (DHFR), hygromycin-B-phosphotransferase (HPH), thymidine kinase (TK), p-galactosidase (lacZ), and xanthine guanine phosphoribosyltransferase (XGPRT), beta-glucuronidase (gus), placental alkaline phosphatase (PLAP), and secreted embryonic alkaline phosphatase (SEAP). Enzyme tags are used with their cognate substrate. The terms also include chemiluminescent labels such as luminol, isoluminol, acridinium esters, and peroxyoxalate and bioluminescent proteins such as firefly luciferase, bacterial luciferase, Renilla luciferase, and aequorin. The terms also include isotopic labels, including radioactive and non-radioactive isotopes, such as, ³H, ²H, ¹²⁰I, ¹²³I, ¹²⁴I, ¹²⁵I, ¹³¹I, ³⁵S, ¹¹C, ¹³C, ¹⁴C, ³²P, ¹⁵N, ¹³N, ¹¹⁰In, ¹¹¹In, ¹⁷⁷Lu, ¹⁸F, ⁵²Fe, ⁶²Cu, ⁶⁴Cu, ⁶⁷Cu, ⁶⁷Ga, ⁶⁸Ga, ⁸⁶Y, ⁹⁰Y, ⁸⁹Zr, ^{94m}Tc, ⁹⁴Tc, ^{99m}Tc, ¹⁵⁴Gd, ¹⁵⁵Gd, ¹⁵⁶Gd, ¹⁵⁷Gd, ¹⁵⁸Gd, ¹⁵O, ¹⁸⁶Re, ¹⁸⁸Re, ⁵¹M, ^{52m}Mn, ⁵⁵Co, ⁷²As, ⁷⁵Br, ⁷⁶Br, ^{82m}Rb, and ⁸³Sr. The terms also include color-coded microspheres of known fluorescent light intensities (see e.g., microspheres with xMAP technology produced by Luminex (Austin, Tex.); microspheres containing quantum dot nanocrystals, for example, containing different ratios and combinations of quantum dot colors (e.g., Qdot nanocrystals produced by Life Technologies (Carlsbad, Calif.); glass coated metal nanoparticles (see e.g., SERS nanotags produced by Nanoplex Technologies, Inc. (Mountain View, Calif.); barcode materials (see e.g., sub-micron sized striped metallic rods such as Nanobarcodes produced by Nanoplex Technologies, Inc.), encoded microparticles with colored bar codes (see e.g., CellCard produced by Vitra Bioscience, vitrabio.com), glass microparticles with digital holographic code images (see e.g., CyVera microbeads produced by Illumina (San Diego, Calif.), near infrared (NIR) probes, and nanoshells. The terms also include contrast agents such as ultrasound contrast agents (e.g. SonoVue microbubbles comprising sulfur hexafluoride, Optison microbubbles comprising an albumin shell and octafluoropropane gas core, Levovist microbubbles comprising a lipid/galactose shell and an air core, Perflexane lipid microspheres comprising perfluorocarbon microbubbles, and Perflutren lipid microspheres comprising octafluoropropane encapsulated in an outer lipid shell), magnetic resonance imaging (MRI) contrast agents (e.g., gadodiamide, gadobenic acid, gadopentetic acid, gadoteridol, gadofosveset, gadoversetamide, gadoxetic acid), and radiocontrast agents, such as for computed tomography (CT), radiography, or fluoroscopy (e.g., diatrizoic acid, metrizoic acid, iodamide, iotalamic acid, ioxitalamic acid, ioglicic acid, acetrizoic acid, iocarmic acid, methiodal, diodone, metrizamide, iohexol, ioxaglic acid, iopamidol, iopromide, iotrolan, ioversol, iopentol, iodixanol, iomeprol, iobitridol, ioxilan, iodoxamic acid, iotroxic acid, ioglycamic acid, adipiodone, iobenzamic acid, iopanoic acid, iocetamic acid, sodium iopodate, tyropanoic acid, and calcium iopodate).

As used herein, a “microbubble” refers to a micron-sized contrast agent composed of a shell and a gas core. The shell may be formed from any suitable material, including, but not limited to, proteins (e.g., albumin), polysaccharides (e.g., galactose), lipids (such as phospholipids), polymers, and combinations thereof. Any suitable gas core can be used in microbubbles, including, but not limited to, air, octafluoropropane, perfluorocarbon, sulfur hexafluoride, or nitrogen. Microbubbles can be used, for example, as contrast agents for ultrasound imaging. The microbubbles oscillate and vibrate when a sonic energy field is applied and reflect ultrasound waves. The gas core determines the echogenecity of the microbubble. The average diameter of a microbubble is typically between about 1 μm and about 25 μm. In general, microbubbles have a diameter ranging between about 1 μm and about 10 μm on average, and more preferably between about 1 μm and 5 μm, 1 μm and 4 μm, 1 μm and 3 μm, 1 μm and about 2 μm, 2 μm and 5 μm, 2 μm and 4 μm, 2 μm and 3 μm, 3 μm and 5 μm, 3 μm and 4 μm, or about 1 μm, 2 μm, 2.5 μm, 3 μm, 3.5 μm, or 4 μm on average.

Following administration to ta patient (such as by intravenous injection), Thy1-targeted microbubbles (e.g., conjugated to an anti-Thy1 single-chain antibody) accumulate at tissue sites that over-express Thy1 causing a local increase in the ultrasound imaging signal. Microbubbles can be used, for example, to detect Thy1 antigen on the surface of pancreatic tumor neovasculature or precancerous lesions that are present in early stage pancreatic cancer.

“Diagnosis” as used herein generally includes determination as to whether a subject is likely affected by a given disease, disorder or dysfunction. The skilled artisan often makes a diagnosis on the basis of one or more diagnostic indicators, i.e., a biomarker, the presence, absence, or amount of which is indicative of the presence or absence of the disease, disorder or dysfunction.

“Prognosis” as used herein generally refers to a prediction of the probable course and outcome of a clinical condition or disease. A prognosis of a patient is usually made by evaluating factors or symptoms of a disease that are indicative of a favorable or unfavorable course or outcome of the disease. It is understood that the term “prognosis” does not necessarily refer to the ability to predict the course or outcome of a condition with 100% accuracy. Instead, the skilled artisan will understand that the term “prognosis” refers to an increased probability that a certain course or outcome will occur; that is, that a course or outcome is more likely to occur in a patient exhibiting a given condition, when compared to those individuals not exhibiting the condition.

The terms “polypeptide” and “protein” refer to a polymer of amino acid residues and are not limited to a minimum length. Thus, peptides, oligopeptides, dimers, multimers, and the like, are included within the definition. Both full length proteins and fragments thereof are encompassed by the definition. The terms also include postexpression modifications of the polypeptide, for example, glycosylation, acetylation, phosphorylation, hydroxylation, and the like. Furthermore, as defined herein, a “polypeptide” refers to a protein which includes modifications, such as deletions, additions and substitutions to the native sequence, so long as the protein maintains the desired activity. These modifications may be deliberate, as through site directed mutagenesis, or may be accidental, such as through mutations of hosts which produce the proteins or errors due to PCR amplification.

By “derivative” is intended any suitable modification of the native polypeptide of interest, of a fragment of the native polypeptide, or of their respective analogs, such as glycosylation, phosphorylation, polymer conjugation (such as with polyethylene glycol), or other addition of foreign moieties, as long as the desired biological activity of the native polypeptide is retained. Methods for making polypeptide fragments, analogs, and derivatives are generally available in the art.

By “fragment” is intended a molecule consisting of only a part of the intact full length sequence and structure. The fragment can include a C-terminal deletion an N-terminal deletion, and/or an internal deletion of the polypeptide. Active fragments of a particular protein or polypeptide will generally include at least about 5-10 contiguous amino acid residues of the full length molecule, preferably at least about 15-25 contiguous amino acid residues of the full length molecule, and most preferably at least about 20-50 or more contiguous amino acid residues of the full length molecule, or any integer between 5 amino acids and the full length sequence, provided that the fragment in question retains biological activity, such as catalytic activity, ligand binding activity, regulatory activity, fluorescence or oligomerization characteristics, as defined herein.

“Substantially purified” generally refers to isolation of a substance (compound, polynucleotide, protein, polypeptide, polypeptide composition) such that the substance comprises the majority percent of the sample in which it resides. Typically in a sample, a substantially purified component comprises 50%, preferably 80%-85%, more preferably 90-95% of the sample. Techniques for purifying polynucleotides and polypeptides of interest are well-known in the art and include, for example, ion-exchange chromatography, affinity chromatography and sedimentation according to density.

By “isolated” is meant, when referring to a polypeptide, that the indicated molecule is separate and discrete from the whole organism with which the molecule is found in nature or is present in the substantial absence of other biological macro molecules of the same type. The term “isolated” with respect to a polynucleotide is a nucleic acid molecule devoid, in whole or part, of sequences normally associated with it in nature; or a sequence, as it exists in nature, but having heterologous sequences in association therewith; or a molecule disassociated from the chromosome.

“Homology” refers to the percent identity between two polynucleotide or two polypeptide molecules. Two nucleic acid, or two polypeptide sequences are “substantially homologous” to each other when the sequences exhibit at least about 50% sequence identity, preferably at least about 75% sequence identity, more preferably at least about 80% 85% sequence identity, more preferably at least about 90% sequence identity, and most preferably at least about 95% 98% sequence identity over a defined length of the molecules. As used herein, substantially homologous also refers to sequences showing complete identity to the specified sequence.

In general, “identity” refers to an exact nucleotide to nucleotide or amino acid to amino acid correspondence of two polynucleotides or polypeptide sequences, respectively. Percent identity can be determined by a direct comparison of the sequence information between two molecules by aligning the sequences, counting the exact number of matches between the two aligned sequences, dividing by the length of the shorter sequence, and multiplying the result by 100. Readily available computer programs can be used to aid in the analysis, such as ALIGN, Dayhoff, M. O. in Atlas of Protein Sequence and Structure M. O. Dayhoff ed., 5 Suppl. 3:353 358, National biomedical Research Foundation, Washington, D.C., which adapts the local homology algorithm of Smith and Waterman Advances in Appl. Math. 2:482 489, 1981 for peptide analysis. Programs for determining nucleotide sequence identity are available in the Wisconsin Sequence Analysis Package, Version 8 (available from Genetics Computer Group, Madison, Wis.) for example, the BESTFIT, FASTA and GAP programs, which also rely on the Smith and Waterman algorithm. These programs are readily utilized with the default parameters recommended by the manufacturer and described in the Wisconsin Sequence Analysis Package referred to above. For example, percent identity of a particular nucleotide sequence to a reference sequence can be determined using the homology algorithm of Smith and Waterman with a default scoring table and a gap penalty of six nucleotide positions.

Another method of establishing percent identity is to use the MPSRCH package of programs copyrighted by the University of Edinburgh, developed by John F. Collins and Shane S. Sturrok, and distributed by IntelliGenetics, Inc. (Mountain View, Calif.). From this suite of packages the Smith Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non redundant, GenBank+EMBL+DDBJ+PDB+GenBank CDS translations+Swiss protein+Spupdate+PIR. Details of these programs are readily available.

Alternatively, homology can be determined by hybridization of polynucleotides under conditions which form stable duplexes between homologous regions, followed by digestion with single stranded specific nuclease(s), and size determination of the digested fragments. DNA sequences that are substantially homologous can be identified in a Southern hybridization experiment under, for example, stringent conditions, as defined for that particular system. Defining appropriate hybridization conditions is within the skill of the art. See, e.g., Sambrook et al., supra; DNA Cloning, supra; Nucleic Acid Hybridization, supra.

“Recombinant” as used herein to describe a nucleic acid molecule means a polynucleotide of genomic, cDNA, viral, semisynthetic, or synthetic origin which, by virtue of its origin or manipulation, is not associated with all or a portion of the polynucleotide with which it is associated in nature. The term “recombinant” as used with respect to a protein or polypeptide means a polypeptide produced by expression of a recombinant polynucleotide. In general, the gene of interest is cloned and then expressed in transformed organisms, as described further below. The host organism expresses the foreign gene to produce the protein under expression conditions.

The term “transformation” refers to the insertion of an exogenous polynucleotide into a host cell, irrespective of the method used for the insertion. For example, direct uptake, transduction or f-mating are included. The exogenous polynucleotide may be maintained as a non-integrated vector, for example, a plasmid, or alternatively, may be integrated into the host genome.

“Recombinant host cells”, “host cells,” “cells”, “cell lines,” “cell cultures,” and other such terms denoting microorganisms or higher eukaryotic cell lines cultured as unicellular entities refer to cells which can be, or have been, used as recipients for recombinant vector or other transferred DNA, and include the original progeny of the original cell which has been transfected.

A “coding sequence” or a sequence which “encodes” a selected polypeptide, is a nucleic acid molecule which is transcribed (in the case of DNA) and translated (in the case of mRNA) into a polypeptide in vivo when placed under the control of appropriate regulatory sequences (or “control elements”). The boundaries of the coding sequence can be determined by a start codon at the 5′ (amino) terminus and a translation stop codon at the 3′ (carboxy) terminus. A coding sequence can include, but is not limited to, cDNA from viral, prokaryotic or eukaryotic mRNA, genomic DNA sequences from viral or prokaryotic DNA, and even synthetic DNA sequences. A transcription termination sequence may be located 3′ to the coding sequence.

Typical “control elements,” include, but are not limited to, transcription promoters, transcription enhancer elements, transcription termination signals, polyadenylation sequences (located 3′ to the translation stop codon), sequences for optimization of initiation of translation (located 5′ to the coding sequence), and translation termination sequences.

“Operably linked” refers to an arrangement of elements wherein the components so described are configured so as to perform their usual function. Thus, a given promoter operably linked to a coding sequence is capable of effecting the expression of the coding sequence when the proper enzymes are present. The promoter need not be contiguous with the coding sequence, so long as it functions to direct the expression thereof. Thus, for example, intervening untranslated yet transcribed sequences can be present between the promoter sequence and the coding sequence and the promoter sequence can still be considered “operably linked” to the coding sequence.

“Encoded by” refers to a nucleic acid sequence which codes for a polypeptide sequence, wherein the polypeptide sequence or a portion thereof contains an amino acid sequence of at least 3 to 5 amino acids, more preferably at least 8 to 10 amino acids, and even more preferably at least 15 to 20 amino acids from a polypeptide encoded by the nucleic acid sequence.

“Expression cassette” or “expression construct” refers to an assembly which is capable of directing the expression of the sequence(s) or gene(s) of interest. An expression cassette generally includes control elements, as described above, such as a promoter which is operably linked to (so as to direct transcription of) the sequence(s) or gene(s) of interest, and often includes a polyadenylation sequence as well. Within certain embodiments of the invention, the expression cassette described herein may be contained within a plasmid construct. In addition to the components of the expression cassette, the plasmid construct may also include, one or more selectable markers, a signal which allows the plasmid construct to exist as single stranded DNA (e.g., a M13 origin of replication), at least one multiple cloning site, and a “mammalian” origin of replication (e.g., a SV40 or adenovirus origin of replication).

“Purified polynucleotide” refers to a polynucleotide of interest or fragment thereof which is essentially free, e.g., contains less than about 50%, preferably less than about 70%, and more preferably less than about at least 90%, of the protein with which the polynucleotide is naturally associated. Techniques for purifying polynucleotides of interest are well-known in the art and include, for example, disruption of the cell containing the polynucleotide with a chaotropic agent and separation of the polynucleotide(s) and proteins by ion-exchange chromatography, affinity chromatography and sedimentation according to density.

The term “transfection” is used to refer to the uptake of foreign DNA by a cell. A cell has been “transfected” when exogenous DNA has been introduced inside the cell membrane. A number of transfection techniques are generally known in the art. See, e.g., Graham et al. (1973) Virology, 52:456, Sambrook et al. (2001) Molecular Cloning, a laboratory manual, 3rd edition, Cold Spring Harbor Laboratories, New York, Davis et al. (1995) Basic Methods in Molecular Biology, 2nd edition, McGraw-Hill, and Chu et al. (1981) Gene 13:197. Such techniques can be used to introduce one or more exogenous DNA moieties into suitable host cells. The term refers to both stable and transient uptake of the genetic material, and includes uptake of peptide- or antibody-linked DNAs.

A “vector” is capable of transferring nucleic acid sequences to target cells (e.g., viral vectors, non-viral vectors, particulate carriers, and liposomes). Typically, “vector construct,” “expression vector,” and “gene transfer vector,” mean any nucleic acid construct capable of directing the expression of a nucleic acid of interest and which can transfer nucleic acid sequences to target cells. Thus, the term includes cloning and expression vehicles, as well as viral vectors.

The terms “variant”, “analog”, and “mutein” refer to biologically active derivatives of the reference molecule that retain desired activity, such as fluorescence or oligomerization characteristics. In general, the terms “variant” and “analog” refer to compounds having a native polypeptide sequence and structure with one or more amino acid additions, substitutions (generally conservative in nature) and/or deletions, relative to the native molecule, so long as the modifications do not destroy biological activity and which are “substantially homologous” to the reference molecule as defined below. In general, the amino acid sequences of such analogs will have a high degree of sequence homology to the reference sequence, e.g., amino acid sequence homology of more than 50%, generally more than 60%-70%, even more particularly 80%-85% or more, such as at least 90%-95% or more, when the two sequences are aligned. Often, the analogs will include the same number of amino acids but will include substitutions, as explained herein. The term “mutein” further includes polypeptides having one or more amino acid-like molecules including but not limited to compounds comprising only amino and/or imino molecules, polypeptides containing one or more analogs of an amino acid (including, for example, unnatural amino acids, etc.), polypeptides with substituted linkages, as well as other modifications known in the art, both naturally occurring and non-naturally occurring (e.g., synthetic), cyclized, branched molecules and the like. The term also includes molecules comprising one or more N-substituted glycine residues (a “peptoid”) and other synthetic amino acids or peptides. (See, e.g., U.S. Pat. Nos. 5,831,005; 5,877,278; and 5,977,301; Nguyen et al., Chem. Biol. (2000) 7:463-473; and Simon et al., Proc. Natl. Acad. Sci. USA (1992) 89:9367-9371 for descriptions of peptoids). Methods for making polypeptide analogs and muteins are known in the art and are described further below.

As explained above, analogs generally include substitutions that are conservative in nature, i.e., those substitutions that take place within a family of amino acids that are related in their side chains. Specifically, amino acids are generally divided into four families: (1) acidic—aspartate and glutamate; (2) basic—lysine, arginine, histidine; (3) non-polar—alanine, valine, leucine, isoleucine, proline, phenylalanine, methionine, tryptophan; and (4) uncharged polar—glycine, asparagine, glutamine, cysteine, serine threonine, and tyrosine. Phenylalanine, tryptophan, and tyrosine are sometimes classified as aromatic amino acids. For example, it is reasonably predictable that an isolated replacement of leucine with isoleucine or valine, an aspartate with a glutamate, a threonine with a serine, or a similar conservative replacement of an amino acid with a structurally related amino acid, will not have a major effect on the biological activity. For example, the polypeptide of interest may include up to about 5-10 conservative or non-conservative amino acid substitutions, or even up to about 15-25 conservative or non-conservative amino acid substitutions, or any integer between 5-25, so long as the desired function of the molecule remains intact. One of skill in the art may readily determine regions of the molecule of interest that can tolerate change by reference to Hopp/Woods and Kyte-Doolittle plots, well known in the art.

By “anti-tumor activity” is intended a reduction in the rate of cell proliferation, and hence a decline in growth rate of an existing tumor or in a tumor that arises during therapy, and/or destruction of existing neoplastic (tumor) cells or newly formed neoplastic cells, and hence a decrease in the overall size of a tumor during therapy. Such activity can be assessed using animal models.

The term “survival” as used herein means the time from the first dose of a Thy-targeted therapeutic agent (e.g., a bioconjugate comprising an anti-Thy1 single-chain antibody conjugated to an anti-cancer therapeutic agent) to the time of death.

By “therapeutically effective dose or amount” of a Thy1-targeted therapeutic agent (e.g., a bioconjugate comprising an anti-Thy1 single-chain antibody conjugated to an anti-cancer therapeutic agent) is intended an amount that, when administered as described herein, brings about a positive therapeutic response, such as an amount that has anti-tumor activity, inhibits metastasis, or increases survival of a subject treated for a pancreatic cancer. The exact amount required will vary from subject to subject, depending on the species, age, and general condition of the subject, the severity of the condition being treated, the particular drug or drugs employed, mode of administration, and the like. An appropriate “effective” amount in any individual case may be determined by one of ordinary skill in the art using routine experimentation, based upon the information provided herein.

The term “tumor response” as used herein means a reduction or elimination of all measurable lesions. The criteria for tumor response are based on the WHO Reporting Criteria [WHO Offset Publication, 48-World Health Organization, Geneva, Switzerland, (1979)]. Ideally, all uni- or bidimensionally measurable lesions should be measured at each assessment. When multiple lesions are present in any organ, such measurements may not be possible and, under such circumstances, up to 6 representative lesions should be selected, if available.

The term “complete response” (CR) as used herein means a complete disappearance of all clinically detectable malignant disease, determined by 2 assessments at least 4 weeks apart.

The term “partial response” (PR) as used herein means a 50% or greater reduction from baseline in the sum of the products of the longest perpendicular diameters of all measurable disease without progression of evaluable disease and without evidence of any new lesions as determined by at least two consecutive assessments at least four weeks apart. Assessments should show a partial decrease in the size of lytic lesions, recalcifications of lytic lesions, or decreased density of blastic lesions.

### EMBODIMENTS

Before describing in detail, it is to be understood that this invention is not limited to particular formulations or process parameters as such may, of course, vary. It is also to be understood that the terminology used herein is for the purpose of describing particular embodiments of the invention only, and is not intended to be limiting.

Although a number of methods and materials similar or equivalent to those described herein can be used in practice, certain preferred materials and methods are described herein.

It is shown herein that an anti-Thy1 single-chain antibody can be used for detection and diagnosis of pancreatic cancer (Example 1). The anti-Thy1 single-chain antibody selectively binds to Thy1 antigen, which is overexpressed on pancreatic tumor neovasculature and precancerous lesions, and is capable of detecting pancreatic cancer even at the earliest stages of the disease. The anti-Thy1 single-chain antibody may be conjugated to various diagnostic agents, such as contrast agents, photoactive agents, or detectable labels, for use in medical imaging and detection of pancreatic tumors. In addition, the anti-Thy1 single-chain antibody may be conjugated to therapeutic agents for targeted treatment of pancreatic cancer.

In order to further an understanding of the invention, a more detailed discussion is provided below regarding anti-Thy1 single-chain antibodies and their use in detection and diagnosis of pancreatic cancer as well as medical imaging and therapeutics.

### Anti-THY Single-Chain Antibodies

In one aspect, the invention includes anti-Thy1 single-chain antibodies capable of specifically binding to Thy1 antigen, which is overexpressed on pancreatic tumor neovasculature and precancerous lesions. The anti-Thy1 single chain antibodies of the invention selectively bind to cancerous pancreatic tissue and not to normal or benign pancreatic tissue and can be used in diagnosing pancreatic cancer as well as differentiating early stage pancreatic cancer from chronic pancreatitis. In particular, such anti-Thy1 single chain antibodies are useful for detecting and diagnosing early-stage pancreatic ductal adenocarcinoma.

Single-chain antibodies comprise an antibody heavy chain variable domain (VH) and a light-chain variable domain (VL) joined together by a flexible peptide linker (e.g., typically 10-25 amino acids in length). Changes can be made to the length and sequence of the peptide linker as long as the single-chain antibody retains Thy1 antigen binding affinity. Advantages of using single-chain antibodies include that they retain the antigen-binding properties of natural full-length antibodies, but lacking the Fc domain, are smaller, have better tumor penetration, and do not stimulate Fc-mediated immune effector functions.

In certain embodiments, the anti-Thy1 single-chain antibody comprises the amino acid sequence of SEQ ID NO:1, or variants thereof comprising an amino acid sequence displaying at least about 80-100% sequence thereto, including any percent identity within this range, such as 81, 82, 83, 84, 85, 86, 87, 88, 89, 90, 91, 92, 93, 94, 95, 96, 97, 98, or 99% sequence identity thereto, or Thy1-binding fragments thereof that bind to essentially the same epitope as or compete for binding to Thy1 with a full-length anti-Thy single-chain antibody. In one embodiment, the single chain antibody binds to human Thy1. Preferably, the anti-Thy1 single-chain antibody binds to human Thy1 with a dissociation constant (Kd) of less than or equal to 3 nM, and more preferably, with a Kd of less than or equal to 1 nM.

A nucleotide sequence encoding an anti-Thy1 single-chain antibody can be constructed using manual or automated nucleotide synthesis, cloned into an expression construct using standard recombinant DNA methods, and introduced into a cell to express the coding sequence, as described below. Alternatively, anti-Thy1 single-chain antibodies can be produced directly using, for example, filamentous phage technology (Verhaar et al., Int. J Cancer 61, 497-501, 1995; Nicholls et al., J. Immunol. Meth. 165, 81-91, 1993).

The anti-Thy1 single-chain antibodies can be purified by methods well known in the art. For example, anti-Thy1 single-chain antibodies can be affinity purified by passage through a column with immobilized Thy1 antigen. The bound anti-Thy1 single-chain antibodies can then be eluted from the column using a buffer with a high salt concentration.

The methods described herein for detection and diagnosis of pancreatic cancer with anti-Thy1 single-chain antibodies may be used in individuals who have not yet been diagnosed (for example, preventative screening), or who have been diagnosed, or who are suspected of having pancreatic cancer (e.g., display one or more characteristic symptoms, presence of pancreatic tumors), or who are at risk of developing pancreatic cancer (e.g., have precancerous lesions). The methods may also be used to detect various stages of progression or severity of disease (e.g., pancreatic precancerous lesions, tumor growth, or metastasis). The methods may also be used to detect the response of the disease to prophylactic or therapeutic treatments or other interventions. The methods can furthermore be used to help the medical practitioner in determining prognosis (e.g., worsening, status-quo, partial recovery, or complete recovery) of the patient, and the appropriate course of action, resulting in either further treatment or observation, or in discharge of the patient from the medical care center.

### Bioconjugation of Anti-Thy1 Single-Chain Antibodies

Anti-Thy1 single-chain antibody bioconjugates may comprise one or more diagnostic or therapeutic agents, or a combination thereof. An anti-Thy1 single-chain antibody may be attached to diagnostic and/or therapeutic agents in a variety of manners. For example, an agent may be attached at the N-terminus, C-terminus, at both the N-terminus and C-terminus, and/or internally, for example, at a residue in the peptide linker between the VH and VL domains. Diagnostic and/or therapeutic agents may be connected directly to the single-chain antibody, or an attached Glycine₅-linker with a C-terminal Cysteine, or indirectly through an intervening linker and or chelating agent (e.g., for metal labeling such as with a radionuclide or paramagnetic metal ion).

Conjugation of single-chain antibodies can be performed using methods well-known in the art. For a discussion of bioconjugation techniques, see, e.g., Chemistry of Bioconjugates: Synthesis, Characterization, and Biomedical Applications (R. Narain ed., Wiley, 2014), G. T. Hermanson Bioconjugate Techniques (Academic Press, 3^{rd} edition, 2013), and Bioconjugation Protocols: Strategies and Methods (Methods in Molecular Biology, S. S. Mark ed., Humana Press, 2^{nd} edition, 2011), van Vught et al. (2014) Comput Struct Biotechnol J. 9:e201402001; Gao et al. (2016) Curr Cancer Drug Targets. May 12 [Epub ahead of print]; Massa et al. (2016) Expert Opin Drug Deliv 13:1-15; Yeh et al. (2015) PLoS One 10(7):e0129681; Freise et al. (2015) Mol Immunol. 67(2 Pt A):142-152; herein incorporated by reference in their entireties.

For example, imaging and/or therapeutic agents can be conjugated to the side chain ε-amine of lysine residues or the free thiol of cysteine residues. In particular, reactions of cysteine thiols with maleimides are commonly used for bioconjugation of proteins. Maleimide-functionalized imaging probes and compounds to facilitate bioconjugation for various imaging modalities are commercially available from a number of companies (e.g., ThermoFisher Scientific (Waltham, Mass.), GE Healthcare Life Sciences (Pittsburgh, Pa.), SigmaAldrich (St. Louis, Mo.), and CHEMATECH (Dijon, France)), including, for example, maleimide lipid derivatives and maleimide albumin derivatives, which can be incorporated into a microbubble shell for ultrasound imaging, maleimide fluorescent dye derivatives for fluorescence imaging, maleimide chelating agent derivatives for binding metals such as radionuclides and paramagnetic cations, and maleimide gold nanoparticle derivatives, which can be used in a variety of ways including as detection agents for electron microscopy and surface enhanced Raman spectroscopy, enhancement agents for radiotherapy, photothermal agents for surface plasmon resonance, and delivery agents for attached drugs or other therapeutic agents.

In certain embodiments, the anti-Thy1 single-chain antibody is engineered to include an N-terminal or C-terminal cysteine residue providing a free thiol group to facilitate conjugation to a reagent comprising a functional group that is reactive with thiols. In other embodiments, a cysteine is incorporated into the linker peptide to allow attachment of reagents to the linker region between the VH and VL domains. Alternatively, additional cysteine residues may be introduced into the single-chain antibody, for example, by site-directed mutagenesis to allow attachment at other sites. A site of attachment away from the antigen binding site should be chosen to avoid interfering with Thy1 targeting of the bioconjugate. In one embodiment, a diagnostic or therapeutic agent is conjugated to the N-terminal cysteine of a single chain antibody comprising the sequence of SEQ ID NO:1.

An alternative bioconjugation method uses click chemistry. Click chemistry reactions include the Huisgen 1,3-dipolar cycloaddition copper catalyzed reaction (Tornoe et al., 2002, J Organic Chem 67:3057-64), cycloaddition reactions such as Diels-Alder reactions, nucleophilic substitution reactions (especially to small strained rings like epoxy and aziridine compounds), reactions involving formation of urea compounds, and reactions involving carbon-carbon double bonds, such as alkynes in thiol-yne reactions. See, e.g., Kolb et al., 2004, Angew Chem Int Ed 40:3004-31; Evans, 2007, Aust J Chem 60:384-95; Millward et al. (2013) Integr Biol (Camb) 5(1):87-95), Lallana et al. (2012) Pharm Res 29(1):1-34, Gregoritza et al. (2015) Eur J Pharm Biopharm. 97(Pt B):438-453, Musumeci et al. (2015) Curr Med Chem. 22(17):2022-2050, McKay et al. (2014) Chem Biol21(9):1075-1101, Ulrich et al. (2014) Chemistry 20(1):34-41, Pasini (2013) Molecules 18(8):9512-9530, and Wangler et al. (2010) Curr Med Chem. 17(11):1092-1116; herein incorporated by reference in their entireties.

### Thy1-Targeted Imaging Agents

Anti-Thy1 single-chain antibodies can be conjugated to diagnostic agents (e.g., probes or detection agents) suitable for various imaging modalities, including, but not limited to, ultrasound imaging (UI), positron emission tomography (PET), single photon emission computed tomography (SPECT), magnetic resonance imaging (MRI), computed tomography (CT), optical imaging (OI), photoacoustic imaging (PI), or fluorescence imaging. Conjugation of a diagnostic agent comprising a detectable moiety or label to an anti-Thy1 single-chain antibody localizes the diagnostic agent to cancerous or pre-cancerous cells expressing the Thy1 antigen. Useful diagnostic agents that can be used in the practice of the invention include, but are not limited to, contrast agents, photoactive agents, radioisotopes, nonradioactive isotopes, dyes, fluorescent compounds or proteins, chemiluminescent compounds, bioluminescent proteins, enzymes, and enhancing agents (e.g., paramagnetic ions).

In certain embodiments, the anti-Thy1 single-chain antibody is conjugated to a contrast agent. Exemplary contrast agents include ultrasound contrast agents (e.g. SonoVue microbubbles comprising sulphur hexafluoride, Optison microbubbles comprising an albumin shell and octafluoropropane gas core, Levovist microbubbles comprising a lipid/galactose shell and an air core, Perflexane lipid microspheres comprising perfluorocarbon microbubbles, and Perflutren lipid microspheres comprising octafluoropropane encapsulated in an outer lipid shell), magnetic resonance imaging (MRI) contrast agents (e.g., gadodiamide, gadobenic acid, gadopentetic acid, gadoteridol, gadofosveset, gadoversetamide, gadoxetic acid), and radiocontrast agents, such as for computed tomography (CT), radiography, or fluoroscopy (e.g., diatrizoic acid, metrizoic acid, iodamide, iotalamic acid, ioxitalamic acid, ioglicic acid, acetrizoic acid, iocarmic acid, methiodal, diodone, metrizamide, iohexol, ioxaglic acid, iopamidol, iopromide, iotrolan, ioversol, iopentol, iodixanol, iomeprol, iobitridol, ioxilan, iodoxamic acid, iotroxic acid, ioglycamic acid, adipiodone, iobenzamic acid, iopanoic acid, iocetamic acid, sodium iopodate, tyropanoic acid, and calcium iopodate).

In one embodiment, an anti-Thy1 single-chain antibody is conjugated to a microbubble that can be used as a contrast agent for ultrasound imaging. Microbubbles are composed of a shell encapsulating a gas core. The shell may be formed from any suitable material, including but not limited to proteins (e.g., albumin), polysaccharides (e.g., galactose), lipids (such as phospholipids), polymers, surfactants, and combinations thereof. Any suitable gas core can be used in microbubbles, including, but not limited to, air, octafluoropropane, perfluorocarbon, sulfur hexafluoride, or nitrogen. The microbubbles oscillate and vibrate when a sonic energy field is applied and reflect ultrasound waves. The gas core determines the echogenicity (i.e., the ability of an object to reflect ultrasound waves) of the microbubble.

The average diameter of a microbubble is typically between about 1 μm and about 25 μm. In general, microbubbles have a diameter ranging between about 1 μm and about 10 μm on average, and more preferably between about 1 μm and 5 μm, 1 μm and 4 μm, 1 μm and 3 μm, 1 μm and about 2 μm, 2 μm and 5 μm, 2 μm and 4 μm, 2 μm and 3 μm, 3 μm and 5 μm, 3 μm and 4 μm, or about 1 μm, 2 μm, 2.5 μm, 3 μm, 3.5 μm, or 4 μm on average.

Various types of microbubbles are commercially available, including but not limited to OPTISON microbubbles (made by GE Healthcare), the first microbubble approved by the Food and Drug Administration (FDA), which have an albumin shell and an octafluoropropane (C₃F₈) gas core; LEVOVIST microbubbles (made by Schering AG), the second FDA-approved microbubble, which have a palmitic acid/galactose shell and an air core; ALBUNEX microbubbles (made by Molecular Biosystems), which have an albumin shell and an air core; SONOVUE microbubbles (made by Bracco Diagnostics, Inc.), which have a sulfur hexafluoride (SF₆) gas core that is stabilized in aqueous dispersion of a monolayer of phospholipids; SONOZOID microbubbles (made by Schering AG), which have a perfluorocarbon gas core and a lipid shell; SONOVIST microbubbles (made by Schering AG), which have a cyanoacrylate polymer shell and an air core; DEFINITY microbubbles (made by DuPont Pharmaceuticals), which have a lipid/surfactant shell and an octafluoropropane (C₃F₈) gas core; and CARDIOSPHERE microbubbles (made by POINT Biomedical Corporation), which have a polyactide polymer shell and a nitrogen gas core.

Microbubbles have a high degree of echogenicity. The echogenicity difference between the gas in the microbubbles and the soft tissue surroundings of the body is large. Thus, ultrasonic imaging using microbubble contrast agents enhances the ultrasound backscatter, or reflection of the ultrasound waves, to produce a unique sonogram with increased contrast due to the high echogenicity difference.

Anti-Thy1 single-chain antibodies can be attached to the shell surface of microbubbles. Following administration to a patient (such as by intravenous injection), microbubbles carrying the anti-Thy1 single-chain antibodies (i.e., Thy1-targeted microbubbles) accumulate at tissue sites that overexpress Thy1 resulting in a local increase in the ultrasound imaging signal. Microbubbles stay predominantly within the vascular compartment after intravenous injection. Thus, microbubbles can be used, for example, to detect Thy1 antigen on the surface of pancreatic tumor neovasculature or precancerous lesions that are present in early stage pancreatic cancer.

The surface of a microbubble can be functionalized in any suitable manner for binding of anti-Thy1 single-chain antibodies. For example, the microbubble surface can be functionalized with maleimide to permit conjugation of a cysteine thiol of the single-chain antibody to the microbubble surface. Alternatively, the microbubble surface can be coated with streptavidin to allow binding of biotinylated anti-Thy1 single-chain antibodies. Any other suitable binding pair can be similarly used, as will be apparent to those of skill in the art.

In other embodiments, the diagnostic agent is a radioactive metal, paramagnetic ion, or other diagnostic cation. In this case, the single-chain antibody can be conjugated to a chelating group for binding cations. Exemplary chelating agents include ethylenediaminetetraacetic acid (EDTA), diethylenetriaminepentaacetic acid (DTPA), 1,4,7,10-tetraazacyclododecane-1,4,7,10-tetraacetic acid (DOTA), 1,4,7-triazacyclononane-N,N′,N″-triacetic acid (NOTA), NETA, p-bromoacetamido-benzyl-tetraethylaminetetraacetic acid (TETA), porphyrins, polyamines, crown ethers, bis-thiosemicarbazones, polyoximes, and like groups known to be useful for this purpose. Chelates are coupled to the single-chain antibodies using standard chemistries, which then can be used to bind diagnostic isotopes such as ¹²⁵I, ¹³¹I, ¹²³I, ¹²⁴I, ⁶²Cu, ⁶⁴Cu, ¹⁸F, ¹¹¹In, ⁶⁷Ga, ⁶⁸Ga, ^{99m}Tc, ²²³Ra, ¹¹C, ¹³N, ¹⁵O, and ⁷⁶Br for radioimaging. The same chelates, when complexed with non-radioactive metals, such as manganese, iron and gadolinium are useful for MRI, when used along with the anti-Thy1 antibodies of the invention.

Diagnostic agents comprising ¹⁸F or ¹¹C can be used in PET imaging. For example, an anti-Thy1 single-chain antibody can be isotopically labeled with ¹⁸F or ¹¹C or conjugated to ¹⁸F or ¹¹C-labeled compounds for use in PET imaging.

### Administration of Thy1-Targeted Imaging Agents

Preferably, a detectably effective amount of a Thy1-targeted imaging agent (e.g., an anti-Thy1 single-chain antibody conjugated to a diagnostic agent) is administered to a subject; that is, an amount that is sufficient to yield an acceptable image using the imaging equipment that is available for clinical use. A detectably effective amount of the Thy1-targeted imaging agent may be administered in more than one injection if needed. The detectably effective amount of the Thy1-targeted imaging agent needed for an individual may vary according to factors such as the degree of binding of the imaging agent to pancreatic tissue, the age, sex, and weight of the individual, and the particular medical imaging method used. Optimization of such factors is within the level of skill in the art.

Imaging with Thy1-targeted imaging agents can be used in assessing efficacy of therapeutic drugs in treating pancreatic cancer. For example, images can be acquired after treatment with an anti-cancer therapy to determine if the individual is responding to treatment. In a subject with pancreatic cancer, imaging with a Thy1-targeted imaging agent can be used to evaluate whether a tumor is shrinking or growing. Further, the extent of cancerous disease (stage of cancer progression) can be determined to aid in determining prognosis and evaluating optimal strategies for treatment (e.g., surgery, radiation, or chemotherapy).

Additionally, Thy1-targeted imaging agents can be used in image-guided surgery. Pancreatic cells or tissue of interest can be contacted with a Thy1-targeted imaging agent, such that the Thy1-targeted imaging agent binds to any Thy1 antigen present on the surface of cells or tissue (e.g., Thy1 overexpressed on pancreatic tumor neovasculature, cancerous cells, or precancerous lesions). Imaging of tissues labeled with the Thy1-targeted imaging agent in this way can be used, for example, for detection of pathology, tumor margin delineation, evaluation of the completeness of resection, and evaluation of the efficacy of treatment.

### Thy1-Targeted Therapeutic Agents

The anti-Thy1 single-chain antibodies of the invention can also be used to target therapeutic agents to the location of Thy1-expressing pancreatic tumors, cancerous cells, or precancerous lesions to directly treat pancreatic cancer in a subject. Anti-Thy1 single-chain antibodies can be conjugated to one or more therapeutic agents, such as, but not limited to, drugs, toxins, radioisotopes, immunomodulators, angiogenesis inhibitors, therapeutic enzymes, and cytotoxic or pro-apoptotic agents for treatment of pancreatic cancer. In addition, such Thy1-targeted therapeutic agents can be used in combination with any other anti-cancer treatment, including, but not limited to, surgery, radiation therapy, chemotherapy, hormonal therapy, immunotherapy, or biologic therapy.

For example, an anti-Thy1 single-chain antibody can be conjugated to one or more chemotherapeutic agents such as, but not limited to, abitrexate, adriamycin, adrucil, amsacrine, asparaginase, anthracyclines, azacitidine, azathioprine, bicnu, blenoxane, busulfan, bleomycin, camptosar, camptothecins, carboplatin, carmustine, cerubidine, chlorambucil, cisplatin, cladribine, cosmegen, cytarabine, cytosar, cyclophosphamide, cytoxan, dactinomycin, docetaxel, doxorubicin, daunorubicin, ellence, elspar, epirubicin, etoposide, fludarabine, fluorouracil, fludara, gemcitabine, gemzar, hycamtin, hydroxyurea, hydrea, idamycin, idarubicin, ifosfamide, ifex, irinotecan, Ianvis, leukeran, leustatin, matulane, mechlorethamine, mercaptopurine, methotrexate, mitomycin, mitoxantrone, mithramycin, mutamycin, myleran, mylosar, navelbine, nipent, novantrone, oncovin, oxaliplatin, paclitaxel, paraplatin, pentostatin, platinol, plicamycin, procarbazine, purinethol, ralitrexed, taxotere, taxol, teniposide, thioguanine, tomudex, topotecan, valrubicin, velban, vepesid, vinblastine, vindesine, vincristine, vinorelbine, VP-16, and vumon.

Alternatively or additionally, an anti-Thy1 single-chain antibody can be conjugated to, one or more tyrosine-kinase inhibitors, such as Imatinib mesylate (Gleevec, also known as STI-571), Gefitinib (Iressa, also known as ZD1839), Erlotinib (marketed as Tarceva), Sorafenib (Nexavar), Sunitinib (Sutent), Dasatinib (Sprycel), Lapatinib (Tykerb), Nilotinib (Tasigna), and Bortezomib (Velcade); Janus kinase inhibitors, such as tofacitinib; ALK inhibitors, such as crizotinib; Bcl-2 inhibitors, such as obatoclax and gossypol; PARP inhibitors, such as Iniparib and Olaparib; PI3K inhibitors, such as perifosine; VEGF Receptor 2 inhibitors, such as Apatinib; AN-152 (AEZS-108) doxorubicin linked to [D-Lys(6)]-LHRH; Braf inhibitors, such as vemurafenib, dabrafenib, and LGX818; MEK inhibitors, such as trametinib; CDK inhibitors, such as PD-0332991 and LEE011; Hsp90 inhibitors, such as salinomycin; and/or small molecule drug conjugates, such as Vintafolide; serine/threonine kinase inhibitors, such as Temsirolimus (Torisel), Everolimus (Afinitor), Vemurafenib (Zelboraf), Trametinib (Mekinist), and Dabrafenib (Tafinlar).

In another example, the anti-Thy1 single-chain antibody can be conjugated to a toxin. The toxin can be of animal, plant or microbial origin. Exemplary toxins include Pseudomonas exotoxin, ricin, abrin, Staphylococcal enterotoxin-A, pokeweed antiviral protein, gelonin, diphtheria toxin, and Pseudomonas endotoxin.

In a further example, the anti-Thy1 single-chain antibody can be conjugated to an immunomodulator, such as a cytokine, a lymphokine, a monokine, a stem cell growth factor, a lymphotoxin (LT), a hematopoietic factor, a colony stimulating factor (CSF), an interferon (IFN), parathyroid hormone, thyroxine, insulin, proinsulin, relaxin, prorelaxin, follicle stimulating hormone (FSH), thyroid stimulating hormone (TSH), luteinizing hormone (LH), hepatic growth factor, prostaglandin, fibroblast growth factor, prolactin, placental lactogen, OB protein, a transforming growth factor (TGF), such as TGF-α or TGF-β, insulin-like growth factor (IGF), erythropoietin, thrombopoietin, a tumor necrosis factor (TNF) such as TNF-α or TNF-β, a mullerian-inhibiting substance, mouse gonadotropin-associated peptide, inhibin, activin, vascular endothelial growth factor, integrin, granulocyte-colony stimulating factor (G-CSF), granulocyte macrophage-colony stimulating factor (GM-CSF), an interferon such as interferon-α, interferon-β, or interferon-γ, S1 factor, an interleukin (IL) such as IL-1, IL-1cc, IL-2, IL-3, IL-4, IL-5, IL-6, IL-7, IL-8, IL-9, IL-10, IL-11, IL-12, IL-13, IL-14, IL-15, IL-16, IL-17, IL-18 IL-21 or IL-25, LIF, kit-ligand, FLT-3, angiostatin, thrombospondin, endostatin, and LT.

In another embodiment, the anti-Thy1 single-chain antibody is conjugated to a radioactive isotope. Particularly useful therapeutic radionuclides include, but are not limited to ¹¹¹In, ¹⁷⁷Lu, ²¹²Bi, ²¹³Bi, ²¹¹At, ⁶²Cu, ⁶⁴Cu, ⁶⁷Cu, ⁹⁰Y, ¹²³I, ¹²⁵I, ¹³¹I, ³²P, ³³P, ⁷⁷Br, ⁴⁷Sc, ¹¹¹Ag, ⁶⁷Ga, ¹⁴²Pr, ¹⁵³Sm, ¹⁶¹Tb, ¹⁶⁶Dy, ¹⁶⁶Ho, ¹⁸⁶Re, ¹⁸⁸Re, ¹⁸⁹Re, ²¹²Pb, ²²³Ra, ²²⁵Ac, ⁵⁹Fe, ⁷⁵Se, ⁷⁷As, ⁸⁹Sr, ⁹⁹Mo, ¹⁰⁵Rh, ¹⁰⁹Pd, ¹⁴³Pr, ¹⁴⁹Pm, ¹⁶⁹Er, ¹⁹⁴Ir, ¹⁹⁸Au, and ¹⁹⁹Au.

In certain embodiments, the therapeutic radionuclide has a decay energy in the range of 20 to 6,000 keV (e.g., 60 to 200 keV for an Auger emitter, 100-2,500 keV for a beta emitter, and 4,000-6,000 keV for an alpha emitter). In one embodiment, the radionuclide is an Auger-emitter (e.g., Co-58, Ga-67, Br-80m, Tc-99m, Rh-103m, Pt-109, In-111, Sb-119, 1-125, Ho-161, Os-189m and Ir-192). In another embodiment, the radionuclide is an alpha-emitter (e.g., Dy-152, At-211, Bi-212, Ra-223, Rn-219, Po-215, Bi-211, Ac-225, Fr-221, At-217, Bi-213 and Fm-255).

Additional therapeutic radioisotopes include ¹¹C, ¹³N, ¹⁵O, ⁷⁵Br, ¹⁹⁸Au, ²²⁴Ac, ¹²⁶I, ¹³³I, ⁷⁷Br, ^{113m}In, ⁹⁵Ru, ⁹⁷Ru, ¹⁰³Ru, ¹⁰⁵Ru, ¹⁰⁷Hg, ²⁰³Hg, ^{121m}Te, ^{122m}Te, ¹⁶⁵Tm, ¹⁶⁷Tm, ¹⁶⁸Tm, ¹⁹⁷Pt, ¹⁰⁹Pd, ¹⁰⁵Rh, ¹⁴²Pr, ¹⁴³Pr, ¹⁶¹Tb, ¹⁶⁶Ho, ¹⁹⁹Au, ⁵⁷Co ⁵¹Cr, ⁵⁹Fe, ⁷⁵Se, ²⁰¹Tl, ²²⁵Ac, ⁷⁶Br, ¹⁶⁹Yb, and the like.

Anti-Thy1 single-chain antibodies may also be conjugated to a boron addend-loaded carrier for thermal neutron activation therapy. For example, boron addends such as carboranes, can be attached to anti-Thy1 single-chain antibodies. Carboranes can be prepared with carboxyl functions on pendant side chains, as is well-known in the art. Attachment of carboranes to a carrier, such as aminodextran, can be achieved by activation of the carboxyl groups of the carboranes and condensation with amines on the carrier. The intermediate conjugate is then conjugated to the anti-Thy1 single-chain antibody. After administration of the anti-Thy1 single chain antibody conjugate, a boron addend is activated by thermal neutron irradiation and converted to radioactive atoms which decay by alpha-emission to produce highly toxic, short-range effects.

### Pharmaceutical Compositions

Bioconjugates of an anti-Thy1 single-chain antibody (e.g., conjugated to one or more diagnostic agents or therapeutic agents, or a combination thereof) can be formulated into pharmaceutical compositions optionally comprising one or more pharmaceutically acceptable excipients. Exemplary excipients include, without limitation, carbohydrates, inorganic salts, antimicrobial agents, antioxidants, surfactants, buffers, acids, bases, and combinations thereof. Excipients suitable for injectable compositions include water, alcohols, polyols, glycerine, vegetable oils, phospholipids, and surfactants. A carbohydrate such as a sugar, a derivatized sugar such as an alditol, aldonic acid, an esterified sugar, and/or a sugar polymer may be present as an excipient. Specific carbohydrate excipients include, for example: monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol, sorbitol (glucitol), pyranosyl sorbitol, myoinositol, and the like. The excipient can also include an inorganic salt or buffer such as citric acid, sodium chloride, potassium chloride, sodium sulfate, potassium nitrate, sodium phosphate monobasic, sodium phosphate dibasic, and combinations thereof.

A composition of the invention can also include an antimicrobial agent for preventing or deterring microbial growth. Nonlimiting examples of suitable antimicrobial agents include benzalkonium chloride, benzethonium chloride, benzyl alcohol, cetylpyridinium chloride, chlorobutanol, phenol, phenylethyl alcohol, phenylmercuric nitrate, thimersol, and combinations thereof.

An antioxidant can be present in the composition as well. Antioxidants are used to prevent oxidation, thereby preventing the deterioration of the anti-Thy1 single-chain antibody bioconjugate (e.g., conjugated to one or more diagnostic agents or therapeutic agents, or a combination thereof), or other components of the preparation. Suitable antioxidants include, for example, ascorbyl palmitate, butylated hydroxyanisole, butylated hydroxytoluene, hypophosphorous acid, monothioglycerol, propyl gallate, sodium bisulfite, sodium formaldehyde sulfoxylate, sodium metabisulfite, and combinations thereof.

A surfactant can be present as an excipient. Exemplary surfactants include: polysorbates, such as “Tween 20” and “Tween 80,” and pluronics such as F68 and F88 (BASF, Mount Olive, N.J.); sorbitan esters; lipids, such as phospholipids such as lecithin and other phosphatidylcholines, phosphatidylethanolamines (although preferably not in liposomal form), fatty acids and fatty esters; steroids, such as cholesterol; chelating agents, such as EDTA; and zinc and other such suitable cations.

Acids or bases can be present as an excipient in the composition. Nonlimiting examples of acids that can be used include those acids selected from the group consisting of hydrochloric acid, acetic acid, phosphoric acid, citric acid, malic acid, lactic acid, formic acid, trichloroacetic acid, nitric acid, perchloric acid, phosphoric acid, sulfuric acid, fumaric acid, and combinations thereof. Examples of suitable bases include, without limitation, bases selected from the group consisting of sodium hydroxide, sodium acetate, ammonium hydroxide, potassium hydroxide, ammonium acetate, potassium acetate, sodium phosphate, potassium phosphate, sodium citrate, sodium formate, sodium sulfate, potassium sulfate, potassium fumerate, and combinations thereof.

The amount of the anti-Thy1 single-chain antibody bioconjugate (e.g., when contained in a drug delivery system) in the composition will vary depending on a number of factors, but will optimally be a therapeutically effective dose when the composition is in a unit dosage form or container (e.g., a vial). A therapeutically effective dose can be determined experimentally by repeated administration of increasing amounts of the composition in order to determine which amount produces a clinically desired endpoint.

The amount of any individual excipient in the composition will vary depending on the nature and function of the excipient and particular needs of the composition. Typically, the optimal amount of any individual excipient is determined through routine experimentation, i.e., by preparing compositions containing varying amounts of the excipient (ranging from low to high), examining the stability and other parameters, and then determining the range at which optimal performance is attained with no significant adverse effects. Generally, however, the excipient(s) will be present in the composition in an amount of about 1% to about 99% by weight, preferably from about 5% to about 98% by weight, more preferably from about 15 to about 95% by weight of the excipient, with concentrations less than 30% by weight most preferred. These foregoing pharmaceutical excipients along with other excipients are described in “Remington: The Science & Practice of Pharmacy”, 19th ed., Williams & Williams, (1995), the “Physician's Desk Reference”, 52nd ed., Medical Economics, Montvale, N.J. (1998), and Kibbe, A. H., Handbook of Pharmaceutical Excipients, 3rd Edition, American Pharmaceutical Association, Washington, D.C., 2000.

The compositions encompass all types of formulations and in particular those that are suited for injection, e.g., powders or lyophilates that can be reconstituted with a solvent prior to use, as well as ready for injection solutions or suspensions, dry insoluble compositions for combination with a vehicle prior to use, and emulsions and liquid concentrates for dilution prior to administration. Examples of suitable diluents for reconstituting solid compositions prior to injection include bacteriostatic water for injection, dextrose 5% in water, phosphate buffered saline, Ringer's solution, saline, sterile water, deionized water, and combinations thereof. With respect to liquid pharmaceutical compositions, solutions and suspensions are envisioned. Additional preferred compositions include those for oral, ocular, or localized delivery.

The pharmaceutical preparations herein can also be housed in a syringe, an implantation device, or the like, depending upon the intended mode of delivery and use. Preferably, the compositions comprising an anti-Thy1 single-chain antibody bioconjugate are in unit dosage form, meaning an amount of a conjugate or composition of the invention appropriate for a single dose, in a premeasured or pre-packaged form.

The compositions herein may optionally include one or more additional agents, such as drugs for treating cancer or other medications used to treat a subject for a condition or disease. Compounded preparations may include an anti-Thy1 single-chain antibody bioconjugate (e.g., conjugated to one or more diagnostic agents or therapeutic agents, or a combination thereof) and one or more drugs for treating pancreatic cancer, such as, but not limited to, chemotherapy, immunotherapy, biologic or targeted therapy agents. Alternatively, such agents can be contained in a separate composition from the composition comprising an anti-Thy1 single-chain antibody bioconjugate and co-administered concurrently, before, or after the composition comprising the anti-Thy1 single-chain antibody bioconjugate of the invention.

### Administration of Thy1-Targeted Therapeutic Agents

The methods of the invention can be used for treating a subject for pancreatic cancer. Thus, Thy1-targeted therapeutic agents comprising an anti-Thy1 single-chain antibody conjugated to an anti-cancer therapeutic agent (or Thy1-targeted theranostic agent also conjugated to a diagnostic agent) can be used to treat, for example, pancreatic cancer expressing the Thy1 antigen, including a tumor, cancer, or metastasis that is progressing, worsening, stabilized or in remission as well as pancreatic precancerous lesions.

At least one therapeutically effective cycle of treatment with a Thy1-targeted therapeutic agent (e.g., an anti-Thy1 single-chain antibody conjugated to an anti-cancer therapeutic agent) will be administered to a subject for treatment of pancreatic cancer. By “therapeutically effective dose or amount” of a Thy1-targeted therapeutic agent is intended an amount that when administered brings about a positive therapeutic response with respect to treatment of an individual for cancer. Of particular interest is an amount of a Thy1-targeted therapeutic agent that provides an anti-tumor effect, as defined herein. By “positive therapeutic response” is intended the individual undergoing the treatment according to the invention exhibits an improvement in one or more symptoms of the cancer for which the individual is undergoing therapy.

Thus, for example, a “positive therapeutic response” would be an improvement in the disease in association with the therapy, and/or an improvement in one or more symptoms of the disease in association with the therapy. Therefore, for example, a positive therapeutic response would refer to one or more of the following improvements in the disease: (1) reduction in tumor size; (2) reduction in the number of cancer cells; (3) inhibition (i.e., slowing to some extent, preferably halting) of tumor growth; (4) inhibition (i.e., slowing to some extent, preferably halting) of cancer cell infiltration into peripheral organs; (5) inhibition (i.e., slowing to some extent, preferably halting) of tumor metastasis; and (6) some extent of relief from one or more symptoms associated with the cancer. Such therapeutic responses may be further characterized as to degree of improvement. Thus, for example, an improvement may be characterized as a complete response. By “complete response” is documentation of the disappearance of all symptoms and signs of all measurable or evaluable disease confirmed by physical examination, laboratory, ultrasound, nuclear, radiographic studies (i.e., CT (computer tomography), and/or MRI (magnetic resonance imaging)), and other non-invasive procedures repeated for all initial abnormalities or sites positive at the time of entry into the study. Alternatively, an improvement in the disease may be categorized as being a partial response. By “partial response” is intended a reduction of greater than 50% in the sum of the products of the perpendicular diameters of all measurable lesions when compared with pretreatment measurements.

In certain embodiments, multiple therapeutically effective doses of compositions comprising a Thy1-targeted therapeutic agent (e.g., an anti-Thy1 single-chain antibody conjugated to an anti-cancer therapeutic agent), and/or one or more other therapeutic agents, such as other drugs for treating cancer, or other medications will be administered. The compositions are typically, although not necessarily, administered orally, via injection (subcutaneously, intravenously, or intramuscularly), by infusion, or locally. Additional modes of administration are also contemplated, such as intra-arterial, intraperitoneal, pulmonary, nasal, topical, transdermal, intralesion, intrapleural, intraparenchymatous, rectal, transdermal, transmucosal, intrathecal, pericardial, intra-arterial, intraocular, and so forth. When administering the anti-Thy1 single-chain antibody by injection, the administration may be by continuous infusion or by single or multiple boluses.

The preparations according to the invention are also suitable for local treatment. In a particular embodiment, a composition of the invention is used for localized delivery of a Thy1-targeted therapeutic agent for the treatment of pancreatic cancer. For example, compositions may be administered directly into a pancreatic tumor or cancerous cells. Administration may be by perfusion through a regional catheter or direct intralesional injection.

The pharmaceutical preparation can be in the form of a liquid solution or suspension immediately prior to administration, but may also take another form such as a syrup, cream, ointment, tablet, capsule, powder, gel, matrix, suppository, or the like. The pharmaceutical compositions comprising a Thy1-targeted therapeutic agent and other agents may be administered using the same or different routes of administration in accordance with any medically acceptable method known in the art.

In another embodiment, the pharmaceutical compositions comprising a Thy1-targeted therapeutic agent and/or other agents are administered prophylactically, e.g., to prevent cancer progression in pancreatic tissue. Such prophylactic uses will be of particular value for subjects with a potentially precancerous or premalignant condition (e.g., precancerous lesions, dysplasia or benign neoplasia), or who have a genetic predisposition to developing pancreatic cancer.

In another embodiment of the invention, the pharmaceutical compositions comprising a Thy1-targeted therapeutic agent and/or other agents are in a sustained-release formulation, or a formulation that is administered using a sustained-release device. Such devices are well known in the art, and include, for example, transdermal patches, and miniature implantable pumps that can provide for drug delivery over time in a continuous, steady-state fashion at a variety of doses to achieve a sustained-release effect with a non-sustained-release pharmaceutical composition.

The invention also provides a method for administering a conjugate comprising a Thy1-targeted therapeutic agent as provided herein to a patient suffering from pancreatic cancer that is responsive to treatment with a Thy1-targeted therapeutic agent contained in the conjugate or composition. The method comprises administering, via any of the herein described modes, a therapeutically effective amount of the conjugate or drug delivery system, preferably provided as part of a pharmaceutical composition. The method of administering may be used to treat any cancer that is responsive to treatment with a Thy1-targeted therapeutic agent. More specifically, the compositions herein are effective in treating pancreatic cancer.

Those of ordinary skill in the art will appreciate which conditions a Thy1-targeted therapeutic agent can effectively treat. The actual dose to be administered will vary depending upon the age, weight, and general condition of the subject as well as the severity of the condition being treated, the judgment of the health care professional, and conjugate being administered. Therapeutically effective amounts can be determined by those skilled in the art, and will be adjusted to the particular requirements of each particular case.

Generally, a therapeutically effective amount will range from about 0.50 mg to 5 grams of a Thy1-targeted therapeutic agent inhibitor daily, more preferably from about 5 mg to 2 grams daily, even more preferably from about 7 mg to 1.5 grams daily. Preferably, such doses are in the range of 10-600 mg four times a day (QID), 200-500 mg QID, 25-600 mg three times a day (TID), 25-50 mg TID, 50-100 mg TID, 50-200 mg TID, 300-600 mg TID, 200-400 mg TID, 200-600 mg TID, 100 to 700 mg twice daily (BID), 100-600 mg BID, 200-500 mg BID, or 200-300 mg BID. The amount of compound administered will depend on the potency of the specific Thy1-targeted therapeutic agent and the magnitude or effect desired and the route of administration.

A purified Thy1-targeted therapeutic agent (again, preferably provided as part of a pharmaceutical preparation) can be administered alone or in combination with one or more other therapeutic agents, such as chemotherapy, immunotherapy, biologic or targeted therapy agents, or other medications used to treat a particular condition or disease according to a variety of dosing schedules depending on the judgment of the clinician, needs of the patient, and so forth. The specific dosing schedule will be known by those of ordinary skill in the art or can be determined experimentally using routine methods. Exemplary dosing schedules include, without limitation, administration five times a day, four times a day, three times a day, twice daily, once daily, three times weekly, twice weekly, once weekly, twice monthly, once monthly, and any combination thereof. Preferred compositions are those requiring dosing no more than once a day.

A Thy1-targeted therapeutic agent can be administered prior to, concurrent with, or subsequent to other agents. If provided at the same time as other agents, the Thy1-targeted therapeutic agent can be provided in the same or in a different composition. Thus, the Thy1-targeted therapeutic agent and other agents can be presented to the individual by way of concurrent therapy. By “concurrent therapy” is intended administration to a subject such that the therapeutic effect of the combination of the substances is caused in the subject undergoing therapy. For example, concurrent therapy may be achieved by administering a dose of a pharmaceutical composition comprising a Thy1-targeted therapeutic agent and a dose of a pharmaceutical composition comprising at least one other agent, such as another drug for treating cancer, which in combination comprise a therapeutically effective dose, according to a particular dosing regimen. Similarly, the Thy1-targeted therapeutic agent and one or more other therapeutic agents can be administered in at least one therapeutic dose. Administration of the separate pharmaceutical compositions can be performed simultaneously or at different times (i.e., sequentially, in either order, on the same day, or on different days), as long as the therapeutic effect of the combination of these substances is caused in the subject undergoing therapy.

Where a subject undergoing therapy in accordance with the previously mentioned dosing regimens exhibits a partial response, or a relapse following a prolonged period of remission, subsequent courses of concurrent therapy may be needed to achieve complete remission of the disease. Thus, subsequent to a period of time off from a first treatment period, a subject may receive one or more additional treatment periods with the Thy1-targeted therapeutic agent. Such a period of time off between treatment periods is referred to herein as a time period of discontinuance. It is recognized that the length of the time period of discontinuance is dependent upon the degree of tumor response (i.e., complete versus partial) achieved with any prior treatment periods of concurrent therapy with these therapeutic agents.

Additionally, treatment with a Thy1-targeted therapeutic agent may be combined with any other medical treatment for cancer, such as, but not limited to, surgery, radiation therapy, chemotherapy, hormonal therapy, immunotherapy, or molecularly targeted or biologic therapy. Any combination of these other medical treatment methods with a Thy1-targeted therapeutic agent may be used to effectively treat cancer in a subject.

For example, treatment with a Thy1-targeted therapeutic agent may be combined with chemotherapy with one or more chemotherapeutic agents such as, but not limited to, abitrexate, adriamycin, adrucil, amsacrine, asparaginase, anthracyclines, azacitidine, azathioprine, bicnu, blenoxane, busulfan, bleomycin, camptosar, camptothecins, carboplatin, carmustine, cerubidine, chlorambucil, cisplatin, cladribine, cosmegen, cytarabine, cytosar, cyclophosphamide, cytoxan, dactinomycin, docetaxel, doxorubicin, daunorubicin, ellence, elspar, epirubicin, etoposide, fludarabine, fluorouracil, fludara, gemcitabine, gemzar, hycamtin, hydroxyurea, hydrea, idamycin, idarubicin, ifosfamide, ifex, irinotecan, lanvis, leukeran, leustatin, matulane, mechlorethamine, mercaptopurine, methotrexate, mitomycin, mitoxantrone, mithramycin, mutamycin, myleran, mylosar, navelbine, nipent, novantrone, oncovin, oxaliplatin, paclitaxel, paraplatin, pentostatin, platinol, plicamycin, procarbazine, purinethol, ralitrexed, taxotere, taxol, teniposide, thioguanine, tomudex, topotecan, valrubicin, velban, vepesid, vinblastine, vindesine, vincristine, vinorelbine, VP-16, and vumon.

In another example, treatment with a Thy1-targeted therapeutic agent may be combined with targeted therapy with one or more small molecule inhibitors or monoclonal antibodies such as, but not limited to, tyrosine-kinase inhibitors, such as Imatinib mesylate (Gleevec, also known as STI-571), Gefitinib (Iressa, also known as ZD1839), Erlotinib (marketed as Tarceva), Sorafenib (Nexavar), Sunitinib (Sutent), Dasatinib (Sprycel), Lapatinib (Tykerb), Nilotinib (Tasigna), and Bortezomib (Velcade); Janus kinase inhibitors, such as tofacitinib; ALK inhibitors, such as crizotinib; Bcl-2 inhibitors, such as obatoclax and gossypol; PARP inhibitors, such as Iniparib and Olaparib; PI3K inhibitors, such as perifosine; VEGF Receptor 2 inhibitors, such as Apatinib; AN-152 (AEZS-108) doxorubicin linked to [D-Lys(6)]-LHRH; Braf inhibitors, such as vemurafenib, dabrafenib, and LGX818; MEK inhibitors, such as trametinib; CDK inhibitors, such as PD-0332991 and LEE011; Hsp90 inhibitors, such as salinomycin; small molecule drug conjugates, such as Vintafolide; serine/threonine kinase inhibitors, such as Temsirolimus (Torisel), Everolimus (Afinitor), Vemurafenib (Zelboraf), Trametinib (Mekinist), and Dabrafenib (Tafinlar); and monoclonal antibodies, such as Rituximab (marketed as MabThera or Rituxan), Trastuzumab (Herceptin), Alemtuzumab, Cetuximab (marketed as Erbitux), Panitumumab, Bevacizumab (marketed as Avastin), and Ipilimumab (Yervoy).

In a further example, treatment with a Thy1-targeted therapeutic agent may be combined with immunotherapy, including, but not limited to, using any of the following: a cancer vaccine (e.g., Sipuleucel-T), antibody therapy (e.g., Alemtuzumab, Ipilimumab, Ofatumumab, Nivolumab, Pembrolizumab, or Rituximab), cytokine therapy (e.g., interferons, including type I (IFNα and IFNβ), type II (IFNγ) and type III (IFNλ) and interleukins, including interleukin-2 (IL-2)), adjuvant immunochemotherapy (e.g., polysaccharide-K), adoptive T-cell therapy, and immune checkpoint blockade therapy.

### Immunoassays Using Anti-Thy1 Single-Chain Antibodies

Anti-Thy1 single-chain antibodies can also be used for detection of pancreatic cancer in vitro. For example, anti-Thy1 single-chain antibodies can be used to detect the presence of the Thy1 antigen in pancreatic tissue from biopsy samples. Anti-Thy1 single-chain antibodies can also be used to detect and measure the amount of Thy1 antigen in pancreatic tissue samples using immunoassay techniques such as immunohistochemistry (IHC), enzyme-linked immunosorbent assay (ELISA), radioimmunoassays (RIA), “sandwich” immunoassays, fluorescent immunoassays, enzyme multiplied immunoassay technique (EMIT), capillary electrophoresis immunoassays (CEIA) immunoprecipitation assays, and western blotting, the procedures of which are well known in the art (see, e.g., Ausubel et al, eds, 1994, Current Protocols in Molecular Biology, Vol. 1, John Wiley & Sons, Inc., New York, which is incorporated by reference herein in its entirety).

Anti-Thy1 single-chain antibodies may be used in diagnostic assays to detect the presence or for quantification of the Thy1 antigen in a pancreatic tissue sample. Such a diagnostic assay may comprise at least two steps; (i) contacting the Thy1 antigen from a pancreatic tissue sample with an anti-Thy1 single-chain antibody, and (ii) quantifying the antibody bound to the Thy1 antigen. The method may additionally involve a preliminary step of attaching the antibody, either covalently, electrostatically, or reversibly, to a solid support, before subjecting the bound antibody to the sample, as defined above and elsewhere herein.

Various diagnostic assay techniques are known in the art, such as competitive binding assays, direct or indirect sandwich assays and immunoprecipitation assays conducted in either heterogeneous or homogenous phases (Zola, Monoclonal Antibodies: A Manual of Techniques, CRC Press, Inc., (1987), pp 147-158). The anti-Thy1 single-chain antibodies used in the diagnostic assays can be labeled with a detectable moiety. The detectable moiety should be capable of producing, either directly or indirectly, a detectable signal. For example, the detectable moiety may be a radioisotope, such as ³H, ¹⁴C, ³²P, or ¹²⁵I, a fluorescent or chemiluminescent compound, such as fluorescein isothiocyanate, rhodamine, or luciferin, or an enzyme, such as alkaline phosphatase, beta-galactosidase, green fluorescent protein, or horseradish peroxidase. Any method known in the art for conjugating antibodies to the detectable moiety may be employed, including those methods described by Hunter et al., Nature, 144:945 (1962); David et al., Biochem., 13:1014 (1974); Pain et al., J. Immunol. Methods, 40:219 (1981); and Nygren, J. Histochem. and Cytochem., 30:407 (1982).

Immunoassays can be used to determine the presence or absence of Thy1 antigen in a pancreatic tissue sample as well as the quantity of the Thy1 antigen in the sample. If the Thy1 antigen is present in the sample, it will form an antibody-antigen complex with the anti-Thy1 single chain antibody, which specifically binds to the Thy1 antigen under suitable incubation conditions. The amount of the antibody-antigen complex can be determined by comparing to a standard. A standard can be, e.g., a known compound or another protein known to be present in a sample.

Immunohistochemistry can be used to detect Thy1 antigen on cancerous cells of a pancreatic tissue section. For example, immunohistochemical staining with labeled anti-Thy1 single-chain antibodies can be used to detect cancerous cells or precancerous lesions. Antibodies conjugated to enzymes, which catalyze color-producing reactions with chromogenic, fluorogenic, or chemiluminescent substrates (e.g., alkaline phosphatase or peroxidase), are commonly used. Alternatively, immunohistochemical staining can be performed with antibodies conjugated to fluorophores (e.g., fluorescein or rhodamine) to visualize biomarkers. See, e.g., Dabbs Diagnostic Immunohistochemistry: Theranostic and Genomic Applications, Saunders, 3^{rd} edition, 2010; Chu Modern Immunohistochemistry (Cambridge Illustrated Surgical Pathology) Cambridge University Press, 2009; Buchwalow et al. Immunohistochemistry: Basics and Methods, Springer, 1st Edition, 2010; and Ramos-Vara (2011) Methods Mol. Biol. 691:83-96; herein incorporated by reference in their entireties.

Flow cytometry can be used to detect multiple surface and intracellular markers simultaneously in whole cells and to distinguish populations of cells expressing different cellular markers. Typically, whole cells are incubated with antibodies that specifically bind to the markers. The antibodies can be labeled, for example, with a fluorophore, isotope, or quantum dot to facilitate detection of the markers. The cells are then suspended in a stream of fluid and passed through an electronic detection apparatus. (See, e.g., Shapiro Practical Flow Cytometry, Wiley-Liss, 4^{th} edition, 2003; Loken Immunofluorescence Techniques in Flow Cytometry and Sorting, Wiley, 2^{nd} edition, 1990; Flow Cytometry: Principles and Applications, (ed. Macey), Humana Press 1^{st} edition, 2007; herein incorporated by reference in their entireties.)

### Production of Anti-Thy1 Single-Chain Antibodies

Anti-Thy1 single-chain antibodies can be produced in any number of ways, all of which are well known in the art. In one embodiment, the anti-Thy1 single-chain antibodies are generated using recombinant techniques. One of skill in the art can readily determine nucleotide sequences that encode an anti-Thy1 single-chain antibody using standard methodology and the teachings herein. Oligonucleotide probes can be devised based on the known sequences and used to probe genomic or cDNA libraries. The sequences can then be further isolated using standard techniques and, e.g., restriction enzymes employed to truncate the gene at desired portions of the full-length sequence. Similarly, sequences of interest can be isolated directly from cells and tissues containing the same, using known techniques, such as phenol extraction and the sequence further manipulated to produce the desired truncations. See, e.g., Sambrook et al., supra, for a description of techniques used to obtain and isolate DNA.

The sequences encoding anti-Thy1 single-chain antibodies can also be produced synthetically, for example, based on the known sequences. The nucleotide sequence can be designed with the appropriate codons for the particular amino acid sequence desired. The complete sequence is generally assembled from overlapping oligonucleotides prepared by standard methods and assembled into a complete coding sequence. See, e.g., Edge (1981) Nature 292:756; Nambair et al. (1984) Science 223:1299; Jay et al. (1984) J. Biol. Chem. 259:6311; Stemmer et al. (1995) Gene 164:49-53.

Recombinant techniques are readily used to clone sequences encoding anti-Thy1 single-chain antibodies that can then be mutagenized in vitro by the replacement of the appropriate base pair(s) to result in the codon for the desired amino acid. Such a change can include as little as one base pair, effecting a change in a single amino acid, or can encompass several base pair changes. Alternatively, the mutations can be effected using a mismatched primer that hybridizes to the parent nucleotide sequence (generally cDNA corresponding to the RNA sequence), at a temperature below the melting temperature of the mismatched duplex. The primer can be made specific by keeping primer length and base composition within relatively narrow limits and by keeping the mutant base centrally located. See, e.g., Innis et al, (1990) PCR Applications: Protocols for Functional Genomics; Zoller and Smith, Methods Enzymol. (1983) 100:468. Primer extension is effected using DNA polymerase, the product cloned and clones containing the mutated DNA, derived by segregation of the primer extended strand, selected. Selection can be accomplished using the mutant primer as a hybridization probe. The technique is also applicable for generating multiple point mutations. See, e.g., Dalbie-McFarland et al. Proc. Natl. Acad. Sci USA (1982) 79:6409.

Once coding sequences have been isolated and/or synthesized, they can be cloned into any suitable vector or replicon for expression. (See, also, Examples). As will be apparent from the teachings herein, a wide variety of vectors encoding modified polypeptides can be generated by creating expression constructs which operably link, in various combinations, polynucleotides encoding polypeptides having deletions or mutations therein.

Numerous cloning vectors are known to those of skill in the art, and the selection of an appropriate cloning vector is a matter of choice. Single-chain antibodies have been successfully produced in variety of hosts, including plants, yeast, and bacteria (see, e.g., Wang et al. (2010) Protein Expr Purif 72:26-31; Brar et al. (2012) Mol Plant Microbe Interact 25:817-824; Huang et al. (2006) Appl Environ Microbiol 72:7748-7759; herein incorporated by reference).

Examples of recombinant DNA vectors for cloning and host cells which they can transform include the bacteriophage A (E. coli), pBR322 (E. coli), pACYC177 (E. coli), pKT230 (gram-negative bacteria), pGV1106 (gram-negative bacteria), pLAFR1 (gram-negative bacteria), pME290 (non-E. coli gram-negative bacteria), pHV14 (E. coli and Bacillus subtilis), pBD9 (Bacillus), pIJ61 (Streptomyces), pUC6 (Streptomyces), YIp5 (Saccharomyces), YCp19 (Saccharomyces) and bovine papilloma virus (mammalian cells). See, generally, DNA Cloning: Vols. I & II, supra; Sambrook et al., supra; B. Perbal, supra.

Insect cell expression systems, such as baculovirus systems, can also be used and are known to those of skill in the art and described in, e.g., Summers and Smith, Texas Agricultural Experiment Station Bulletin No. 1555 (1987). Materials and methods for baculovirus/insect cell expression systems are commercially available in kit form from, inter alia, Invitrogen, San Diego Calif. (“MaxBac” kit).

Plant expression systems can also be used to produce the anti-Thy1 single-chain antibodies described herein. Generally, such systems use virus-based vectors to transfect plant cells with heterologous genes. For a description of such systems see, e.g., Porta et al., Mol. Biotech. (1996) 5:209-221; and Hackland et al., Arch. Virol. (1994) 139:1-22.

Viral systems, such as a vaccinia based infection/transfection system, as described in Tomei et al., J. Virol. (1993) 67:4017-4026 and Selby et al., J. Gen. Virol. (1993) 74:1103-1113, will also find use. In this system, cells are first transfected in vitro with a vaccinia virus recombinant that encodes the bacteriophage T7 RNA polymerase. This polymerase displays exquisite specificity in that it only transcribes templates bearing T7 promoters. Following infection, cells are transfected with the DNA of interest, driven by a T7 promoter. The polymerase expressed in the cytoplasm from the vaccinia virus recombinant transcribes the transfected DNA into RNA that is then translated into protein by the host translational machinery. The method provides for high level, transient, cytoplasmic production of large quantities of RNA and its translation product(s).

The gene can be placed under the control of a promoter, ribosome binding site (for bacterial expression) and, optionally, an operator (collectively referred to herein as “control” elements), so that the DNA sequence encoding the desired polypeptide is transcribed into RNA in the host cell transformed by a vector containing this expression construction. The coding sequence may or may not contain a signal peptide or leader sequence. Both the naturally occurring signal peptides and heterologous sequences can be used. Leader sequences can be removed by the host in post-translational processing. See, e.g., U.S. Pat. Nos. 4,431,739; 4,425,437; 4,338,397. Such sequences include, but are not limited to, the TPA leader, as well as the honey bee mellitin signal sequence.

Other regulatory sequences may also be desirable which allow for regulation of expression of the protein sequences relative to the growth of the host cell. Such regulatory sequences are known to those of skill in the art, and examples include those which cause the expression of a gene to be turned on or off in response to a chemical or physical stimulus, including the presence of a regulatory compound. Other types of regulatory elements may also be present in the vector, for example, enhancer sequences.

The control sequences and other regulatory sequences may be ligated to the coding sequence prior to insertion into a vector. Alternatively, the coding sequence can be cloned directly into an expression vector that already contains the control sequences and an appropriate restriction site.

In some cases it may be necessary to modify the coding sequence so that it may be attached to the control sequences with the appropriate orientation; i.e., to maintain the proper reading frame. Mutants or analogs may be prepared by the deletion of a portion of the sequence encoding the protein, by insertion of a sequence, and/or by substitution of one or more nucleotides within the sequence. Techniques for modifying nucleotide sequences, such as site-directed mutagenesis, are well known to those skilled in the art. See, e.g., Sambrook et al., supra; DNA Cloning, Vols. I and II, supra; Nucleic Acid Hybridization, supra.

The expression vector is then used to transform an appropriate host cell. A number of mammalian cell lines are known in the art and include immortalized cell lines available from the American Type Culture Collection (ATCC), such as, but not limited to, Chinese hamster ovary (CHO) cells, HeLa cells, baby hamster kidney (BHK) cells, monkey kidney cells (COS), human hepatocellular carcinoma cells (e.g., Hep G2), Vero293 cells, as well as others. Similarly, bacterial hosts such as E. coli, Bacillus subtilis, and Streptococcus spp., will find use with the present expression constructs. Yeast hosts include inter alia, Saccharomyces cerevisiae, Candida albicans, Candida maltosa, Hansenula polymorpha, Kluyveromyces fragilis, Kluyveromyces lactis, Pichia guillerimondii, Pichia pastoris, Schizosaccharomyces pombe and Yarrowia lipolytica. Insect cells for use with baculovirus expression vectors include, inter alia, Aedes aegypti, Autographa californica, Bombyx mori, Drosophila melanogaster, Spodoptera frugiperda, and Trichoplusia ni.

Depending on the expression system and host selected, the anti-Thy1 single-chain antibodies are produced by growing host cells transformed by an expression vector described above under conditions whereby the protein of interest is expressed. The selection of the appropriate growth conditions is within the skill of the art.

In one embodiment, the transformed cells secrete the polypeptide product into the surrounding media. Certain regulatory sequences can be included in the vector to enhance secretion of the protein product, for example using a tissue plasminogen activator (TPA) leader sequence, an interferon (γ or α) signal sequence or other signal peptide sequences from known secretory proteins. The secreted polypeptide product can then be isolated by various techniques described herein, for example, using standard purification techniques such as but not limited to, hydroxyapatite resins, column chromatography, ion-exchange chromatography, size-exclusion chromatography, electrophoresis, HPLC, immunoadsorbent techniques, affinity chromatography, immunoprecipitation, and the like.

Alternatively, the transformed cells are disrupted, using chemical, physical or mechanical means, which lyse the cells yet keep the recombinant polypeptides substantially intact. Intracellular proteins can also be obtained by removing components from the cell wall or membrane, e.g., by the use of detergents or organic solvents, such that leakage of the polypeptides occurs. Such methods are known to those of skill in the art and are described in, e.g., Protein Purification Applications: A Practical Approach, (Simon Roe, Ed., 2001).

For example, methods of disrupting cells include but are not limited to: sonication or ultrasonication; agitation; liquid or solid extrusion; heat treatment; freeze-thaw; desiccation; explosive decompression; osmotic shock; treatment with lytic enzymes including proteases such as trypsin, neuraminidase and lysozyme; alkali treatment; and the use of detergents and solvents such as bile salts, sodium dodecylsulphate, Triton, NP40 and CHAPS. The particular technique used to disrupt the cells is largely a matter of choice and will depend on the cell type in which the polypeptide is expressed, culture conditions and any pre-treatment used.

Following disruption of the cells, cellular debris is removed, generally by centrifugation, and the intracellularly produced polypeptides are further purified, using standard purification techniques such as but not limited to, column chromatography, ion-exchange chromatography, size-exclusion chromatography, electrophoresis, HPLC, immunoadsorbent techniques, affinity chromatography, immunoprecipitation, and the like.

For example, one method for obtaining intracellular polypeptides involves affinity purification, such as by immunoaffinity chromatography using antibodies (e.g., previously generated antibodies), or by affinity chromatography using Thy1 as a ligand. The choice of a suitable affinity resin is within the skill in the art. After affinity purification, the polypeptides can be further purified using conventional techniques well known in the art, such as by any of the techniques described above.

Anti-Thy1 single-chain antibodies can be conveniently synthesized chemically, for example by any of several techniques that are known to those skilled in the peptide art. In general, these methods employ the sequential addition of one or more amino acids to a growing peptide chain. Normally, either the amino or carboxyl group of the first amino acid is protected by a suitable protecting group. The protected or derivatized amino acid can then be either attached to an inert solid support or utilized in solution by adding the next amino acid in the sequence having the complementary (amino or carboxyl) group suitably protected, under conditions that allow for the formation of an amide linkage. The protecting group is then removed from the newly added amino acid residue and the next amino acid (suitably protected) is then added, and so forth. After the desired amino acids have been linked in the proper sequence, any remaining protecting groups (and any solid support, if solid phase synthesis techniques are used) are removed sequentially or concurrently, to render the final polypeptide. By simple modification of this general procedure, it is possible to add more than one amino acid at a time to a growing chain, for example, by coupling (under conditions which do not racemize chiral centers) a protected tripeptide with a properly protected dipeptide to form, after deprotection, a pentapeptide. See, e.g., J. M. Stewart and J. D. Young, Solid Phase Peptide Synthesis (Pierce Chemical Co., Rockford, Ill. 1984) and G. Barany and R. B. Merrifield, The Peptides: Analysis, Synthesis, Biology, editors E. Gross and J. Meienhofer, Vol. 2, (Academic Press, New York, 1980), pp. 3-254, for solid phase peptide synthesis techniques; and M. Bodansky, Principles of Peptide Synthesis, (Springer-Verlag, Berlin 1984) and E. Gross and J. Meienhofer, Eds., The Peptides: Analysis, Synthesis, Biology, Vol. 1, for classical solution synthesis. These methods are typically used for relatively small polypeptides, i.e., up to about 50-100 amino acids in length, but are also applicable to larger polypeptides.

Typical protecting groups include t-butyloxycarbonyl (Boc), 9-fluorenylmethoxycarbonyl (Fmoc) benzyloxycarbonyl (Cbz); p-toluenesulfonyl (Tx); 2,4-dinitrophenyl; benzyl (Bzl); biphenylisopropyloxycarboxy-carbonyl, t-amyloxycarbonyl, isobornyloxycarbonyl, o-bromobenzyloxycarbonyl, cyclohexyl, isopropyl, acetyl, o-nitrophenylsulfonyl and the like.

Typical solid supports are cross-linked polymeric supports. These can include divinylbenzene cross-linked-styrene-based polymers, for example, divinylbenzene-hydroxymethylstyrene copolymers, divinylbenzene-chloromethylstyrene copolymers and divinylbenzene-benzhydrylaminopolystyrenecopolymers.

Polypeptide analogs can also be chemically prepared by other methods such as by the method of simultaneous multiple peptide synthesis. See, e.g., Houghten Proc. Natl. Acad. Sci. USA (1985) 82:5131-5135; U.S. Pat. No. 4,631,211.

### Kits

In yet another aspect, the invention provides kits comprising an anti-Thy1 single-chain antibody that can be used to detect Thy1 antigen on the surface of pancreatic tumor neovasculature, cancerous cells, or precancerous lesions. Such kits can be used for detection, diagnosis, medical imaging, and/or treatment of pancreatic cancer.

In certain embodiments, the anti-Thy1 single-chain antibody comprises an amino acid sequence of SEQ ID NO:1 or a variant thereof comprising an amino acid sequence displaying at least about 80-100% sequence identity thereto, including any percent identity within this range, such as 81, 82, 83, 84, 85, 86, 87, 88, 89, 90, 91, 92, 93, 94, 95, 96, 97, 98, 99% sequence identity, or a Thy1-binding fragment thereof that binds to the same epitope as or competes for binding to Thy1 with the anti-Thy1 single-chain antibody comprising the amino acid sequence of SEQ ID NO:1.

In certain embodiments, the kit comprises a Thy1-targeted imaging agent, therapeutic agent, or theranostic agent. For example, the kit may comprise a bioconjugate of an anti-Thy1 single chain antibody conjugated to one or more imaging agents or therapeutic agents, or a combination thereof.

Compositions can be in liquid form or can be lyophilized. Suitable containers for the compositions include, for example, bottles, vials, syringes, and test tubes. Containers can be formed from a variety of materials, including glass or plastic. A container may have a sterile access port (for example, the container may be an intravenous solution bag or a vial having a stopper pierceable by a hypodermic injection needle).

The kit can further comprise a second container comprising a pharmaceutically-acceptable buffer, such as phosphate-buffered saline, Ringer's solution, or dextrose solution. It can also contain other materials useful to the end-user, including other pharmaceutically acceptable formulating solutions such as buffers, diluents, filters, needles, and syringes or other delivery devices. The delivery device may be pre-filled with the compositions.

The kit can also comprise a package insert containing written instructions for methods of using the compositions comprising the anti-Thy1 antibody or bioconjugates thereof (e.g., Thy1-targeted imaging agents, therapeutic agents, or theranostic agents) for diagnosing and/or treating pancreatic cancer. The instructions may also describe methods of using the compositions to detect and/or image cancerous cells or tumors in pancreatic tissue expressing Thy1, and methods of diagnosing and monitoring disease progression and therapeutic efficacy. The package insert can be an unapproved draft package insert or can be a package insert approved by the Food and Drug Administration (FDA) or other regulatory body.

### EXPERIMENTAL

The following examples are put forth so as to provide those of ordinary skill in the art with a complete disclosure and description, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Early Pancreatic Ductal Adenocarcinoma Detection Using Engineered Anti-Thy1-Single-Chain Antibody-Targeted Contrast Agents

One potential strategy for earlier detection of pancreatic ductal adenocarcinoma (PDAC) involves the screening of moderate and high-risk patients using molecularly-targeted contrast ultrasound agents that bind and amplify the signal of molecular markers expressed on the neovasculature, hence, increasing the sensitivity and specificity of ultrasound imaging in detecting early stage cancer. Rapid and reliable detection of PDAC is a highly attractive aim in medical imaging with potential major benefits for patients. Recently, an extensive proteomic analysis has identified and validated the thymocyte differentiation antigen 1 (Thy1) as a specific marker which is upregulated on the neovasculature of PDAC compared to chronic pancreatitis and normal patients. Thy1, also known as cluster of differentiation 90 or CD90, is a cell-surface glycoprotein that belongs to the immunoglobulin-like supergene family. Thy1 was originally described as a mouse thymocyte differentiation marker, and subsequently, it was found to be expressed in other tissues, including the surface of newly formed blood vessels of colon cancer, glioblastoma, hepatocellular carcinoma, and ovarian cancer tissues. Recently, an in vivo proof-of-principle study illustrated the potential to detect neovasculature of PDAC using a pre-clinical-grade Thy1-targeted ultrasound contrast agent. The development of hybridoma technology has designed a multitude of antibodies and explored for medical purposes. However, the development and production of antibodies against a specific target is challenging, costly, and time-consuming. Alternatively, the evolving engineered protein scaffold using the yeast display technique (EPS) facilitates the development of novel binders to a target of interest.

In this study, the overall goal was to engineer an anti-Thy1 single-chain antibody (anti-Thy1-scFv) with a molecular weight of ˜29 kDa and to develop a novel clinically translatable anti-Thy1-scFv-targeted ultrasound contrast agent. In contrast to antibodies, engineered scFvs are inexpensive to produce in large quantities, because of their relatively small size. The design of anti-Thy1-scFv-targeted contrast agent may pave the way for improving the visualization and earlier detection of PDAC, thus, improving overall survival of patients with PDAC.

### Material and Methods:

Human Thy1-Expressing Vascular Endothelial Cells. Wildtype MILE SVEN 1 mouse vascular endothelial (MS1-WT) cells [CRL2279; American Type Culture Collection (ATCC)] cells were cultured under sterile conditions in Dulbecco's Modified Eagle Medium (ATCC) with FBS at 5% and maintained in a 5% CO₂-humidified atmosphere at 37° C. Cells were transfected with human Thy1 DNA using standard techniques. In brief, the human Thy1 DNA sequence (gi|224589802:c119294246-119288655) was first optimized for mammalian codon usage as described. The transfection of MS1-WT cells with the Thy1-expression vector was performed using lipofectamine 2000 transfection reagent (Life Sciences; Invitrogen), following the recommended manufacturer's standard protocol. MS1 cells stably expressing human Thy1 (MS1_{Thy1}) were grown in DMEM containing 10% fetal bovine serum and 0.4 mg/ml G418 in a 5% CO₂ humidified atmosphere and subcultured prior to confluence using trypsin.

Engineering of an Anti-Thy1 single-chain antibodies. A scFv yeast surface display (YSD) library (2.5×10⁹ diversity), in which EBY100 yeast cells were transformed with pCT surface display vector containing the scFv gene, was screened with a combination of magnetic bead sorting and fluorescence-activated cell sorting (FACS) with recombinant human and mouse Thy1 as described. A single round of yeast isolation consisted of two isolations with streptavidin-magnetic beads conjugated to biotinylated recombinant human and mouse Thy1 (B-Thy1, Thermo Fisher) and one FACS isolation against double-positive yeasts with 500 nM Thy1 and c-Myc. Then, scFv gene fragments were obtained after error-prone polymerase chain reaction (E-PCR) against plasmids isolated from sorted yeasts and new libraries for the next rounds of yeast isolation were constructed by transformation of the fragments and pCT vector, as described. Seven rounds of yeast isolation were performed to complete the screening. After the fourth round of yeast isolation, yeasts were further isolated after double staining with lower concentration of Thy1 and an anti-cMyc antibody (eBioscience) using FACS. For FACS isolation, yeasts were stained with 50 nM B-Thy1 at 4° C. for 1.5 hr. After a simple wash with PBS containing 0.1% BSA (PBSA), yeasts were incubated with streptavidin-APC (1:100 dilution) and chicken anti-cMyc (1:100 dilution) antibodies at 4° C. for 1 hr. Yeasts were then counterstained with Alexa 555-conjugated goat anti-chicken IgY (1:100 dilution) at 4° C. for 40 min. Yeasts of the highest Thy1-binding affinity were isolated with FACS and further cultured in 10 mL of SD-CAA media at 30° C. and 250 rpm for 1 day. After the induction of scFv-YSD, isolated yeast cultures were centrifuged at 5000 rpm for 1 min and placed into SG-CAA media. At final seventh round, the FACS isolations were repeated twice after staining with 10 nM, 5 nM, 3 nM and 1 nM Thy1. The final isolated yeast clone (Thy1-scFv) with the highest affinity was further analyzed, and the plasmid was isolated with a Zymoprep Yeast Plasmid Miniprep II kit. The isolated plasmid of the yeast clone Thy1-scFv was sequenced and subcloned into E. coli.

Affinity measurements of Anti-Thy1 single-chain antibody against human and mouse Thy1. The dissociation constant (KD) of the Anti-Thy1 single-chain antibody were measured using yeast as described. Yeast cells (1×10⁵) transformed with pCT plasmid containing Anti-Thy1 single-chain antibody gene were incubated with 0.01 to 100 nM recombinant human and mouse biotinylated-Thy1 overnight at room temperature. Cells were stained with the anti-myc antibody. After FACS analysis, mean fluorescence values for APC dye and Alexa 555 in double-positive populations of each yeast sample were acquired. KD values were analyzed by determining the ratio of the mean fluorescence intensity of [APC] and fluorescence intensity of [A555] using FACS. The ratio were plotted against the used concentration of Thy1 using Kaleidgraph. The KD was determined using a nonlinear least squares curve.

Purification of Anti-Thy1 single-chain antibody. To construct the scFv expression vector for Anti-Thy1 single-chain antibody, the gene was amplified. The amplified PCR fragment was digested with NcoI and XhoI and ligated into the same sites in the E. coli pET32b expression vector, which contains a 6×His tag and enterokinase sequence in the N-terminus of anti-Thy1-scFv. The scFv ligand Anti-Thy1 single-chain antibody purification was performed in SHuffle T7 E. coli, transformed with bacterial expression vector. A single bacterial colony grown on an LB plate supplemented with ampicillin (50 μg/mL) was inoculated into 5 mL of lysogeny broth (LB) media supplemented with amp. After overnight culture, bacteria were transferred to 1 L of LB media and grown at 30° C. and 250 rpm for 4 hr. Bacteria were further cultured at 30° C. and 250 rpm for 6 hr after 0.5 M isopropyl p-D-1-thiogalactopyranoside (IPTG) was added. The bacterial pellet was harvested via centrifugation at 3,200×g for 10 min, and was resuspended in 3 mL of ice-cold lysis buffer. The supernatant obtained by centrifugation at 12,000×g for 5 min were applied to a HisTrap FF column (GE Healthcare Biosciences, PA) in an AKTA FPLC system (GE Healthcare Biosciences), and 6×His-tag Anti-Thy1 single-chain antibodies were isolated and then lyophilized. The concentration of purified Anti-Thy1 single-chain antibody protein was measured by UV spectrometry after dissolving in PBS. Evaluation of purity of the Anti-Thy1 single-chain antibody was analyzed using SDS-PAGE electrophoresis. 30 μl of each purified protein and 6 μl of 5× reducing SDS loading buffer were added to 1.5 ml tubes and denatured at 96° C. for 5 minutes. The samples were run on SDS-PAGE gel in SDS running buffer at 30 mA for 2 hours. The gel was then stained with Coomassie Brilliant Blue for 1 hour and 3 subsequently destained for at least 12 hours with Coomassie destaining solution. The gel was visualized and analyzed using a BioRad Gel-Doc system.

In vitro binding analysis of purified Thy1-scFv. Thy1-coated magnetic beads were prepared as described above, and analyzed with FACS. Briefly, 66 pmol of biotinylated Thy1 was incubated with 10 μL of streptavidin-magnetic beads in 50 μL of PBSA for 40 min at room temperature. As controls, naked streptavidin-beads and beads coated with the same amount of biotinylated human IgG were prepared. Subsequently, beads were washed with PBSA and incubated with 10 nM purified Anti-Thy1 single-chain antibody for 1.5 hr at room temperature. The Anti-Thy1 single-chain antibody bound to beads were stained with 40 μL of AF647-conjugated anti-his tag antibody (1:100 dilution) and analyzed by FACS. For cell binding assay, MS1-WT and MS1_{Thy1} were stained with an AF647-conjugated anti-his tag antibody-labeled Anti-Thy1 single-chain antibody for 1.5 hr at 4° C. and analyzed with FACS. As a control, primary antibody (rabbit anti-human Thy1, Sigma, 1:100) incubation was performed. After washing in PBS, secondary antibody (anti-rabbit FITC, Jackson Immunolaboratories, 1:500) was added for one hour at 4° C. and analyzed with FACS.

In vitro immunofluorescence staining of human Thy1-expressing murine vascular endothelial cells. To confirm human Thy1 expression on vascular endothelial MS1 cells, immunofluorescence staining of the cells was performed using standard techniques. In brief, MS1-WT and MS1-Thy1 cells were grown on cover slips under standard conditions in DMEM complete growth media for 24 hours; after the media was removed, cells were washed in PBS and fixed in 4% paraformaldehyde in PBS solution for 30 min at room temperature. Cells were then washed in PBS, and 1% bovine serum albumin (BSA) blocking solution was applied for one hour. The biotinylated Anti-Thy1 single-chain antibody (100 nM) incubation was performed for 2h at 4° C. After washing in PBS, secondary antibody (Streptavidin-AF555, ThermoFisher Scientific) was added for one hour at room temperature. Cells were then washed in PBS, counterstained with 4′,6-diamidino-2-phenylindole (DAPI), mounted onto glass slides with anti-fade solution and imaged with an Olympus IX81 system.

While the preferred embodiments of the invention have been illustrated and described, it will be appreciated that various changes can be made therein without departing from the spirit and scope of the invention.

What is claimed is:

1. A single chain antibody selected from the group consisting of:
a single chain antibody comprising an amino acid sequence of SEQ ID NO:1;
a single chain antibody comprising an amino acid sequence having at least 95% identity to the sequence of SEQ ID NO:1, wherein the single chain antibody specifically binds to a thymocyte differentiation antigen 1 (Thy1); and
a single chain antibody comprising a Thy1-binding fragment of the single chain antibody of (a) or (b) that binds to the same epitope as or competes for binding to Thy1 with the single chain antibody of (a) or (b).

2. The single chain antibody of claim 1 comprising the sequence of SEQ ID NO:1.

3. The single chain antibody of claim 1, wherein the single chain antibody binds to human Thy1.

4. The single chain antibody of claim 1, wherein the single chain antibody binds to Thy1 with a dissociation constant (Kd) of less than or equal to 2 nM.

5. A Thy1-targeted imaging agent comprising the single chain antibody of claim 1 conjugated to a diagnostic agent.

6. The Thy1-targeted imaging agent of claim 5, wherein the diagnostic agent is a contrast agent or a photoactive agent.

7. The Thy1-targeted imaging agent of claim 6, wherein the contrast agent is an ultrasound contrast agent, a magnetic resonance imaging (MRI) contrast agent, or a radiocontrast agent.

8. The Thy1-targeted imaging agent of claim 7, wherein the ultrasound contrast agent is a microbubble.

9. The Thy1-targeted imaging agent of claim 5, wherein the diagnostic agent comprises a detectable label.

10. The Thy1-targeted imaging agent of claim 9, wherein the detectable label is a fluorescent label, a radioactive isotopic label, a non-radioactive isotopic label, a chemiluminescent label, a bioluminescent label, a paramagnetic ion, or an enzyme.

11. The Thy1-targeted imaging agent of claim 10, wherein the fluorescent label is selected from the group consisting of a fluorescein derivative, a rhodamine derivative, a coumarin derivative, a cyanine derivative, an acridine derivative, a squaraine derivative, a naphthalene derivative, an oxadiazol derivative, an anthracene derivative, a pyrene derivative, an oxazine derivative, an arylmethine derivative, a tetrapyrrole derivative, and a fluorescent protein.

12. The Thy1-targeted imaging agent of claim 11, wherein the fluorescent protein is selected from the group consisting of green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), TagRFP, Dronpa, Padron, mApple, mCherry, rsCherry, and rsCherryRev.

13. The Thy1-targeted imaging agent of claim 10, wherein the isotopic label is selected from the group consisting of ³H, ²H, ¹²⁰I, ¹²³I, ¹²⁴I, ¹²⁵I, ¹³¹I, ³⁵S, ¹¹C, ¹³C, ¹⁴C, ³²P, ¹⁵N, ¹³N, ¹¹⁰In, ¹¹¹In, ¹⁷⁷Lu, ¹⁸F, ⁵²Fe, ⁶²Cu, ⁶⁴Cu, ⁶⁷Cu, ⁶⁷Ga, ⁶⁸Ga, ⁸⁶Y, ⁹⁰Y, ⁸⁹Zr, ^{94m}Tc, ⁹⁴Tc, ^{99m}Tc, ¹⁵⁴Gd, ¹⁵⁵Gd, ¹⁵⁶Gd, ¹⁵⁷Gd, ¹⁵⁸Gd, ¹⁵O, ¹⁸⁶Re, ¹⁸⁸Re, ⁵¹M, ^{52m}Mn, ⁵⁵Co, ⁷²As, ⁷⁵Br, ⁷⁶Br, ^{82m}Rb, and ⁸³Sr.

14. The Thy1-targeted imaging agent of claim 10, wherein the detectable label comprises a radionuclide selected from the group consisting of a gamma-emitter, a beta-emitter, and a positron-emitter.

15. The Thy1-targeted imaging agent of claim 10, wherein the paramagnetic ion is selected from the group consisting of chromium (III), manganese (II), iron (II), iron (II), cobalt (II), nickel (II), copper (II), neodymium (II), samarium (II), ytterbium (II), gadolinium (II), vanadium (II), terbium (III), dysprosium (III), holmium (III) and erbium (II).

16. The Thy1-targeted imaging agent of claim 10, wherein the anti-Thy1 single chain antibody is further conjugated to an anti-cancer therapeutic agent.

17. The Thy1-targeted imaging agent of claim 16, wherein the anti-cancer therapeutic agent is selected from the group consisting of a cytotoxic agent, a drug, a toxin, a nuclease, a hormone, an immunomodulator, a pro-apoptotic agent, an anti-angiogenic agent, a boron compound, a photoactive agent, and a radioisotope.

18. The Thy1-targeted imaging agent of claim 5, wherein the diagnostic agent is conjugated to an N-terminal cysteine of the single-chain antibody.

19. A composition comprising the Thy1-targeted imaging agent of claim 5.

20. The composition of claim 19, further comprising a pharmaceutically acceptable excipient.

21. The composition of claim 19, further comprising an anti-cancer therapeutic agent.

22. A method of detecting pancreatic cancer or precancerous lesions, the method comprising:
administering a detectably effective amount of the Thy1-targeted imaging agent of claim 5 to a patient suspected of having pancreatic cancer, under conditions wherein the Thy1-targeted imaging agent binds to Thy1 present on pancreatic tumor neovasculature, cancerous cells, or precancerous lesions, if present, in the patient; and
detecting the Thy1-targeted imaging agent bound to the pancreatic tumor neovasculature, cancerous cells, or precancerous lesions, if present, by imaging pancreatic tissue of the patient.

23. The method of claim 22, wherein said imaging is performed using ultrasound imaging (UI), positron emission tomography (PET), single photon emission computed tomography (SPECT), magnetic resonance imaging (MRI), computed tomography (CT), optical imaging (OI), photoacoustic imaging (PI), or fluorescence imaging.

24. The method of claim 22, wherein the pancreatic cancer is pancreatic ductal adenocarcinoma.

25. The method of claim 22, wherein detection of a precancerous lesion indicates the patient is at risk of developing pancreatic cancer.

26. The method of claim 22, wherein the patient is human.

27. A method of imaging pancreatic tissue of a patient suspected of having pancreatic cancer, the method comprising:
contacting pancreatic tissue of the patient with a detectably effective amount of the Thy1-targeted imaging agent of claim 5 under conditions wherein the Thy1-targeted imaging agent binds to Thy1 present on any pancreatic tumor neovasculature, cancerous cells, or precancerous lesions, if present, in the pancreatic tissue; and
imaging pancreatic tissue of the patient, wherein detection of increased binding of the Thy1-targeted imaging agent to the pancreatic tissue of the patient compared to a control indicates that the patient has pancreatic cancer or precancerous lesions.

28. The method of claim 27, wherein the pancreatic tissue is contacted with the Thy1-targeted imaging agent in vivo or in vitro.

29. The method of claim 27, wherein said imaging is performed using ultrasound imaging (UI), positron emission tomography (PET), single photon emission computed tomography (SPECT), magnetic resonance imaging (MRI), computed tomography (CT), optical imaging (OI), photoacoustic imaging (PI), or fluorescence imaging.

30. A method of monitoring progression of pancreatic cancer in a patient, the method comprising:
imaging pancreatic tissue of the patient according to the method of claim 27, wherein a first image is obtained at a first time point and a second image is obtained later at a second time point, wherein detection of increased binding of the Thy1-targeted imaging agent to the pancreatic tissue of the patient at the second time point compared to the first time point indicates that the patient is worsening, wherein detecting decreased binding of the Thy1-targeted imaging agent to the pancreatic tissue of the patient at the second time point compared to the first time point indicates that the patient is improving.

31. The method of claim 30, wherein increased binding of the Thy1-targeted imaging agent to the pancreatic tissue of the patient is associated with growth of a pancreatic tumor or presence of more pancreatic tumors or lesions at the second time point.

32. A method for evaluating the effect of an agent for treating pancreatic cancer in a patient, the method comprising:
imaging pancreatic tissue of the patient according to the method of claim 27 before and after the patient is treated with said agent, wherein detection of increased binding of the Thy1-targeted imaging agent to the pancreatic tissue of the patient after the patient is treated with said agent compared to before the patient is treated with said agent indicates that the patient is worsening, and decreased binding of the Thy1-targeted imaging agent to the pancreatic tissue of the patient after the subject is treated with said agent compared to before the patient is treated with said agent indicates that the patient is improving.

33. A method for monitoring the efficacy of a therapy for treating pancreatic cancer in a patient, the method comprising:
imaging pancreatic tissue of the patient according to the method of claim 27 before and after the subject undergoes said therapy, wherein detection of increased binding of the Thy1-targeted imaging agent to the pancreatic tissue of the patient after the patient undergoes said therapy compared to before the patient undergoes said therapy indicates that the patient is worsening, and decreased binding of the Thy1-targeted imaging agent to the pancreatic tissue of the patient after the patient undergoes said therapy compared to before the patient undergoes said therapy indicates that the patient is improving.

34. A kit comprising the antibody of claim 1 and instructions for using the kit to diagnose pancreatic cancer.

35. A kit comprising the Thy1-targeted imaging agent of claim 5 and instructions for using the kit to diagnose pancreatic cancer.

36. A method of treating a patient suspected of having pancreatic cancer, the method comprising:
receiving information regarding whether or not pancreatic cancer was detected in the patient according to the method of claim 22; and
administering anti-cancer therapy to the subject if pancreatic cancer was detected in the patient.

37. The method of claim 36, wherein the anti-cancer therapy comprises surgery, radiation therapy, chemotherapy, hormonal therapy, immunotherapy, or biologic therapy, or any combination thereof.

38. A Thy1-targeted therapeutic agent comprising the single chain antibody of claim 1 conjugated to an anti-cancer therapeutic agent.

39. The Thy1-targeted therapeutic agent of claim 38, wherein the anti-cancer therapeutic agent is selected from the group consisting of a cytotoxic agent, a drug, a toxin, a nuclease, a hormone, a therapeutic enzyme, an immunomodulator, a pro-apoptotic agent, an angiogenesis inhibitor, a boron compound, a photoactive agent, and a radioisotope.

40. A composition comprising the Thy1-targeted therapeutic agent of claim 38.

41. The composition of claim 40, further comprising a pharmaceutically acceptable excipient.

42. The composition of claim 40, further comprising an anti-cancer therapeutic agent.

43. A kit comprising the Thy1-targeted therapeutic agent of claim 38 and instructions for using the kit to treat pancreatic cancer.

44. An isolated polynucleotide encoding the single chain antibody of claim 1.

45. A recombinant polynucleotide comprising the polynucleotide of claim 44 operably linked to a promoter.

46. A method for producing the single-chain antibody of claim 1, the method comprising:
transforming a host cell with a recombinant polynucleotide comprising a polynucleotide encoding the single-chain antibody of claim 1 operably linked to a promoter;
culturing the transformed host cell under conditions whereby the single-chain antibody is expressed; and
isolating the single-chain antibody from the host cell.


SEQUENCE LISTING (1 sequence)

SEQ ID NO: 1 - protein, 260 aa, Artificial Sequence
  h5-Thy1
CMASASQVQLVQSGAEVKKPGASVKVSCKASGYTFTGYYVHWVRQAPGQGLEWMGWVNPNSGDTNYAQKFQGRVTMTRDTSISTAYMELSGLRSDDTAVYYCARDGDEDWYFDLWGRGTPVTVSSGILGSGGGGSGGGGSGGGGSDIRLTQSPSSLSASIGDRVTITCRASQGISRSLVWYQQKPGKAPRLLIYAASTLQSGVPSRFSGSGSGTDFTLTISSLQPEDFATYYCLQHNTYPFTFGPGTKVDIKSGIPEQKL